(19) United States
(12) Patent Application Publication
(10) Pub. No.: US 2019/0365719 A1
(43) Pub. Date: Dec. 5, 2019

(54) COMBINATION TREATMENTS OF HSP90 INHIBITORS FOR ENHANCING TUMOR IMMUNOGENICITY AND METHODS OF USE THEREOF

(71) Applicants: Massachusetts Institute of Technology, Cambridge, MA (US); Whitehead Institute for Biomedical Research, Cambridge, MA (US); The Brigham and Women's Hospital, Boston, MA (US); President and Fellows of Harvard College, Cambridge, MA (US)

(72) Inventors: Tyler Jacks, Newton, MA (US); Alexander Jaeger, Cambridge, MA (US); Sandro Santagata, West Roxbury, MA (US); Luke Whitesell, Somerville, MA (US); Susan Lindquist, Cambridge, MA (US)

(21) Appl. No.: 16/430,116

(22) Filed: Jun. 3, 2019

Related U.S. Application Data
(60) Provisional application No. 62/679,704, filed on Jun. 1, 2018.

(51) Int. Cl. A61K 31/4196; C07K 16/28

(52) U.S. Cl. CPC A61K 31/4196; A61K 45/06; C07K 16/2818; C07K 16/2827

(57) ABSTRACT

It has been established that exposure to cytotoxic doses of HSP90 inhibitor is broadly immunosuppressive, whereas continuous exposure to low-dosages of the same inhibitor exerts anti-tumor activity. The anti-tumor activity is mediated by the host immune system. Compositions and methods for continuous, low-dose exposure to HSP90 inhibitors in combination with one or more immunostimulatory agents for the treatment of cancer are described. Typically, the HSP90 inhibitor is administered in an amount that is between 1% and 20% of the clinically-determined maximum tolerate dose. The immunostimulatory agent can be administered simultaneously with the HSP90 inhibitor, or at some time before or after the HSP90 inhibitor. Compositions including a sub-toxic dose of HSP90 inhibitor in combination with an immunostimulatory agent in an amount effective to treat cancer are also provided.

### CROSS-REFERENCE TO RELATED APPLICATIONS

This application claims the benefit of and priority to U.S. Provisional Application No. 62/679,704 filed Jun. 1, 2018, which is hereby incorporated by reference in its entirety.

### STATEMENT REGARDING FEDERALLY SPONSORED RESEARCH

This invention was made with government support under Grant No. R01 CA194005 awarded by National Institutes of Health, and under Grant No. P30-259 CA14051 awarded by National Cancer Institute, and under Grant No. W81XWH-14-1-0157 awarded by the Department of Defense. The government has certain rights in the invention.

### FIELD OF INVENTION

The present invention relates to the treatment of cancer and in particular to the application of low-dosages of inhibitors of the heat shock protein HSP90 pathway in combination with immunostimulatory agents for the enhanced killing of tumor cells.

### BACKGROUND OF THE INVENTION

Over the past decade the molecular chaperone heat-shock protein 90 kDa (HSP90) has been extensively investigated as a promising anticancer target. Theoretically, inhibiting the function of chaperones such as HSP90 could simultaneously attack multiple oncoproteins. Sustained inhibition of oncogenic signaling pathways has potential to selectively kill tumor cells harboring driver mutations in oncogenes and overcome the heterogeneity of clinical cancers that frequently enable the emergence of resistance.

However, clinical testing of an array of highly optimized HSP90 inhibitor chemotypes, either alone or in combination with other agents, has been largely disappointing. Despite the central role of HSP90 inhibitors in oncogenic signaling, so far, limited activity associated with poor therapeutic index has prevented any HSP90 inhibitor from advancing to become an approved therapy. This clinical experience suggests the problem arises not from the limitations of any particular drug scaffold, but from an underlying flaw in therapeutic strategy.

Moreover, in the case of HSP90 inhibitors, systemic toxicity rises markedly as the duration of high level, client-depleting drug exposure increases. As a result, unlike the daily or more frequent dosing typical of signaling inhibitors, HSP90 inhibitors have been tested in patients almost universally on intermittent, bolus dosing schedules, with drug administration occurring approximately once or twice weekly. Unfortunately, such episodic challenges to protein homeostasis exemplify the periodic perturbations that the cytoprotective heat-shock response evolved to counteract in guarding the proteome. Moreover, activation of Heat Shock Factor 1 (HSF1), the master transcriptional regulator of this response, has recently emerged in its own right as a powerful enabler of malignancy, affecting far more than just chaperone levels in both cancer cells and the stromal cells that support them.

In view of the collateral damage that inhibiting HSP90 can cause, potentially deleterious effects on antitumor immune function have gone virtually unexplored during the clinical development of HSP90 inhibitors.

Therefore, it is an object of the invention to provide compositions and methods for selectively killing cancer cells through inhibiting the HSP90 pathway without systemic toxicity.

It is also an object of the invention to provide dosage units of HSP90 inhibitors in combination with immunostimulatory agents and methods of using thereof for enhancing the efficacy of anti-cancer therapy with HSP90 inhibitors.

### SUMMARY OF THE INVENTION

It has been established that the constitutive, incomplete inhibition of heat-shock protein 90 (HSP90) resulting from continuous (i.e. daily) administration of HSP90 inhibitors (HSP90i) selectively kills cancer cells in the absence of systemic toxicity. Sub-toxic HSP90 inhibition can produce a steady state plasma level of HSP90i that is approximately 1%, 5%, 10% or 20% the amount of the maximum recommended bolus dose. This dosing gives rise to specific cancer cell killing by tumor-specific immune cells in the absence of systemic toxicity. This dosing regimen is fundamentally distinct from previous clinical exploration of HSP90i where intermittent (i.e. once weekly) high doses (maximum tolerated dose) of HSP90i were administered to completely or almost completely ablate HSP90 function where the primary goal of treatment was to directly kill cancer cells. It has also been established that combining low-dose HSP90i(s) with one or more agents that non-specifically enhances or stimulates the immune system enhances the anti-tumor efficacy of the HSP90i.

The examples demonstrate that a continuous, low-dose administration of HSP90i in combination with an adjuvant of the immune system reduces the viability and proliferation of cancer cells. Specifically, the examples show that the low-dose administration of NVP-HSP990 elicits a specific anti-tumor effect that is dependent upon the expression of MHC class I on tumor cells. The examples also show that the continuous low-dose administration of HSP90i in combination with a single dose of an immune adjuvant increased long-term survival compared with administration of either agent alone.

Pharmaceutical compositions containing an effective amount of the combination of one or more HSP90i(s) and one or more immunostimulatory agent(s) to reduce cancer cell proliferation or reduce cancer cell viability, or reduce both cancer cell viability and proliferation in a subject with cancer are provided. The amount of the HSP90i(s) does not induce systemic toxicity in the subject. In some embodiments, administration of the pharmaceutical composition reduces cancer cell proliferation and/or cancer cell viability in the subject to a greater degree than administering the same amount of the HSP90i(s) alone or the same amount of the immunostimulatory agent(s) alone. In some embodiments, the reduction in cancer cell proliferation or viability in the subject with cancer is more than the additive reduction achieved by administering the HSP90i(s) alone or the immunostimulatory agent(s) alone.

Exemplary classes of HSP90i(s) include benzoquinone ansamycin antibiotics, resorcinol derivatives, purine scaffold HSP90i(s), functional nucleic acid inhibitors of HSP90, inhibitors of one or more co-chaperones of HSP90, and other antibiotic or small molecule HSP90i(s). Typically, the amount of the HSP90i is a low dose that is between 1% and 50% of the amount that is the maximum tolerated dose (MTD) in humans. In a preferred embodiment, the amount of the HSP90i is 5% of the amount that is the maximum tolerated dose (MTD) in humans.

Exemplary classes of immunostimulatory agents include pro-inflammatory molecules, adjuvants, tumor antigens, antagonists of immuno-suppressors, modulators or regulatory T cells (T-regs) and co-stimulatory antibodies. An exemplary immunostimulatory agent is an anti-PD-L1 monoclonal antibody in an amount between 1 mg/kg and 15 mg/kg body weight of the recipient. In some embodiments, the pharmaceutical composition includes an additional active agent, such as an additional anti-cancer agent such as a conventional chemotherapeutic agent that inhibits proliferation of the cancer cells.

Methods of making appropriate dosage formulations including providing dosage units for administration of HSP90i in a low level for daily administration, alone or in combination with an immunostimulatory agent. These may be provided in a single dosage unit or more typically in a kit, so that the HSP90i is administered daily and the immunostimulatory agent at less frequent intervals. The dosage units will typically be formulated as lyophilized or dry powders for resuspension for injection, or in a form for oral administration, although controlled release dosage forms may also be employed.

Methods of treating cancer in a human patient including administering to the patient an effective amount of one or more HSP90i(s), preferably in combination with an effective amount of one or more immunostimulatory agent(s) and, optionally in combination with other therapeutic agents, are provided. Administration of the combination of one or more HSP90i(s) and one or more immunostimulatory agent(s) reduces cancer cell proliferation or viability in the patient to a greater degree than administering to the patient the same amount of HSP90i(s) or the same amount of immunostimulatory agent(s) alone. In some embodiments, the reduction in cancer cell proliferation or viability in the subject with cancer is more than the additive reduction achieved by administering the HSP90i(s) and or the same amount of immunostimulatory agent(s) alone. In some embodiments, the methods administer an amount of HSP90i(s) that does not affect the cancer cells when the HSP90i(s) is administered without co-administration of the immunostimulatory agent(s). In some embodiments, one or more immunostimulatory agent(s) is administered to the subject 1, 2, 3, 4, 5, 6, 8, 10, 12, 18, or 24 hours, 1, 2, 3, 4, 5, 6, or 7 days, or any combination thereof prior to administration of one or more HSP90i(s) to the patient. In other embodiments, one or more HSP90i(s) is administered to the subject 1, 2, 3, 4, 5, 6, 8, 10, 12, 18, or 24 hours, 1, 2, 3, 4, 5, 6, or 7 days, or any combination thereof prior to administration of one or more immunostimulatory agents to the subject.

In some embodiments, the methods include administering to the subject one or more additional active agents, such as an additional anti-cancer agent. In some embodiments, the methods include surgery or radiation therapy.

### BRIEF DESCRIPTION OF THE DRAWINGS

FIGS. 1A-1E are comparisons of immune-related genes in pre and post-treatment with HSP90i (ganetespib) intravenously: FIG. 1A is a schematic representation of the sample collection process. Whole blood was collected at baseline and 20-24 hours post intravenous (IV) HSP90i (ganetespib) prior to total RNA isolation and analysis by NanoString Expression Profiling. FIG. 1B is a Volcano plot of 521 immune-related genes passing Nanostring quality control metrics. Genes functionally related to antigen processing are in darker grey and the top six most significantly downregulated antigen-processing genes are indicated. FIG. 1C is a graph showing Immune Pathway scoring of gene expression data using Nanostring Advanced Analysis of 9 immune-related pathways significantly affected following ganetispib treatment (each point represents an individual patient). FIG. 1D is a graph showing signature scores of the immune-related pathways that were not significantly affected following ganetispib treatment; FIG. 1E and FIG. 1F are dot plots showing expression analysis of the pre and post-treatment with ganetespib samples using a NanoString Codeset for heat-shock response genes at 4 hr (FIG. 1E) and 24 hr (FIG. 1F) post treatment

FIGS. 2A-2B are comparisons of transcript analyzed by RNA-seq of human peripheral blood mononuclear cells (PBMCs) treated with DMSO vehicle or ganetespib for 20 hours. FIG. 2A is a plot of the log-ratio versus mean expression (MA plot) of the transcripts, gray points represent genes that are significantly upregulated and light gray points represent significantly downregulated genes (DEseq2 adjusted p-value<0.05); FIG. 2B is a bar graph showing gene sets and their fold enrichment in vehicle or ganetespib-treated human PBMCs calculated by Gene ontology analysis of RNA-seq data using Panther (pantherdb.org). Dark grey histograms represent significantly (p<0.05) upregulated gene sets and light grey histograms represent significantly downregulated gene sets.

FIGS. 3A-3H are graphs showing continuous low dose administration of HSP90i eliciting an anti-tumor response. FIG. 3A is a line graph showing subcutaneous MC38 tumor growth in C57Bl/6 (Black lines) or NOD-SCID (Red lines) mice were treated via drinking water with vehicle (solid lines) or 0.5 mg/kg/day HSP90i (dotted lines) (n=10 per group). ***p<0.001-3-way ANOVA, Tukey's multiple comparison test; FIG. 3B is a graph showing MC38 tumor weights in C57Bl/6 or NOD-SCID mice 18 days post implantation. **p<0.01, 2-way ANOVA, Tukey's multiple comparison test. FIG. 3C is a graph showing plasma concentration of HSP90i in Bl/6 and NOD-SCID mice measured by quantitative liquid chromatography/mass spectrometry assay (n=10 per group). FIG. 3D is a floating bar graph showing Image quantification of the abundance of CD3+ (n=5) or CD8+ (n=10) cells per tumor area in vehicle or HSP90i-treated mice; FIG. 3E is a graph showing median fluorescence intensity of MHC Class I H2-Kb surface expression on dissociated MC38 tumor cells in vehicle-treated or Hspo90i-treated mice. (n=5 for Veh, n=4 for Hsp90i) *p<0.05 two-tailed, unpaired, Welch's t-test. FIG. 3F is a floating bar graph showing signature scores from Nanostring Pan-Cancer Immunology analysis of data derived from MC38 tumor tissue in vehicle-treated or Hspo90i-treated mice; FIG. 3G is a Volcano plot of Nanostring data shown in FIG. 3F; FIG. 3H is a bar graph showing qRT-PCR analysis of heat-shock gene expression in bulk MC38 tumor tissue of mice treated with vehicle or Hsp90i.

FIGS. 4A-4F are graphs showing low dose administration of HSP90i stimulates antigen presentation of tumor cells. FIG. 4A shows representative flow cytometric histograms showing MHC Class I H2-Kb cell surface expression of MC38 cells treated with vehicle or with HSP90i at 15 nM, 30 nM, and 60 nM concentrations; FIG. 4B is a graph showing MHC Class I H2-Kb cell surface expression based on quantification of 5 independent biological replicates of samples processed as in FIG. 4A. *p<0.05, ****p<0. FIG. 4C is a line graph showing relative MC38 cell number following exposure to increasing concentrations of Hsp90i; Live cell number was assessed with a standard metabolic dye-based (Alamar blue) assay and cell mass assessed by protein content using sulphorhodamine-B (SRB) assay. Representative histograms showing flow cytometric measurement of relative MHC Class I expression on the surface of triple-negative breast cancer (SUM159) (FIG. 4D), melanoma (SKMEL) (FIG. 4E), and lung adenocarcinoma (H838) (FIG. 4F) cells treated with DMSO (Veh) or low dose HSP990 for 72 hours.

FIGS. 5A-5L are graphs showing low dose administration of HSP90i diversifies the antigen repertoire of tumor cells. FIG. 5A is a bar graph showing relative mRNA expression MHC Class I and immunoproteasome gene expression in MC38 cells treated for 72 hours with HSP90i (60 nM) based on qRT-PCR analysis. All treated samples were normalized to DMSO (Veh)=1 (n=3); FIG. 5B is a schematic depiction of the effect of HSP90i treatment on proteasome composition and ONX-0914 mode of action; FIG. 5C is a bar graph showing cell surface expression of MHC Class I on MC38 cells treated with vehicle or HSP90i in combination with DMSO or ONX-0914 (500 nM). Normalized values calculated based on the median fluorescence intensity of the baseline sample for DMSO- or ONX-0914-treated samples (n=3); FIG. 5D is a schematic overview of protocol for MHC Class I peptide profiling; motif analysis for amino acid enrichment of 8-mer (FIG. 5E) and 9-mer peptides (FIG. 5F) identified by mass spectrometry; FIG. 5G is a histogram depicting the size distribution of peptides identified by MHC Class I IP and mass spectrometry; FIG. 5H is a Venn diagram of the number of unique peptides identified in vehicle- and Hsp90i-treated samples. The size of each circle is proportional to the number of unique peptides identified; FIG. 5I and FIG. 5J are Venn diagrams of peptide profiling data derived from the single biological replicates which were used to generate the merged data presented in FIG. 5H; FIG. 5K is a histogram showing the number of unique peptides identified with increasing number of technical MS replicates for Veh- and Hsp90-i treated samples; FIG. 5L is a histogram of peptides identified in both Veh and Hsp90i samples binned according to their log 2 fold change in relative abundance (Hsp90i/Veh) and fit with a non-linear Gaussian distribution (curve). Light grey vertical line indicates x=0 and dark grey vertical line indicates the center of the distribution.

FIGS. 6A-B are graphs showing anti-tumor activity of low-dose HSP90i is dependent on functional MHC Class I expression. Ctrl or B2m KO cells were generated by CRISPR-Cas9 mediated gene editing, and B2m KO cells were confirmed to no longer express MHC-I on the cell surface. FIG. 6A is a line graph showing relative number of viable Ctrl and B2m KO cells measured by standard dye reduction assay following 2-day exposure to increasing concentrations of HSP90i; FIG. 6B is a line graph showing size in mm³ of Ctrl or B2m KO MC38 tumors in mice treated with vehicle (Veh, solid lines) or 0.5 mg/kg/day HSP90i (dotted lines) over 25 days post implantation.

FIG. 7 is a survival curve of MC38 tumor-bearing mice treated with vehicle (black, solid line), HSP90i (black, dotted), CD40/PolyI:C (Adj; grey, solid) or HSP90i+Adj (grey, dotted).

FIGS. 8A-8D are line graphs showing size in mm³ over time of the individual tumors monitored in MC38 tumor-bearing mice treated with vehicle (FIG. 8A), HSP90i (FIG. 8B), Adj (CD40/PolyI:C) (FIG. 8C), or HSP90i+Adj (FIG. 8D) groups to generate the survival analysis presented in FIG. 7. Mice were sacrificed when tumor volume surpassed 1500 mm³ or mice exhibited body condition <=2.

FIG. 9 is a schematic diagram showing a proposed model for HSP90i-enhanced antigen presentation and anti-tumor immunity.

FIG. 10A is a Venn diagram depicting the number of unique MHC-1 peptides that were observed when MHC-1 cells were treated with vehicle (control), IFN-γ, or Hsp90i. FIGS. 10B-10E are bar graphs depicting qRT-PCR analysis of antigen presentation genes (FIG. 10B), antigen processing genes (FIG. 10C), immune checkpoint genes (FIG. 10D), and heat shock inducible genes (FIG. 10E) in MC38 cells following 72 hour treatment with Hsp90i (60 nM) or IFNγ (10 ng/mL). Samples were normalized to DMSO=1 (n=3). FIG. 10F and FIG. 10G shows representative flow cytometric histograms of MHC-I (FIG. 10F) and PD-L1 (FIG. 10G) expression on MC38 cells treated with vehicle control, Hsp90i or IFN-γ. HSP90i is able to rescue the surface expression of MHC-I molecules in cells that lack responsiveness to IFNγ following CRISPR/Cas9 mediated gene editing of the Ifngr. FIG. 10H is a representative flow cytometric histogram showing MHC-I staining of an Hsf1 KO MC38 cell line (sgHsf1.1) treated with vehicle (control), Hsp90i, IFN-γ, or Hsp90i+IFN-γ. Median fluorescence intensity of MHC-I staining is shown on the right of each histogram.

FIG. 11A is a box plot showing tumor burden analysis in KP and KPM mice treated with vehicle (control) or Hsp90i. FIG. 11B is a graph showing the longitudinal analysis of mouse weights with vehicle or Hsp90i in the drinking water. The mice were treated for up to 4 weeks with vehicle control or a low dose of HSP90i. In FIG. 11B, KP and KPM mice were combined for vehicle and control treatment groups.

### DETAILED DESCRIPTION OF THE INVENTION

### I. Definitions

The terms “heat-shock protein 90” and “HSP90” includes each member of the family of heat shock proteins having a mass of about 90 kilo Daltons. For example, in humans the highly conserved HSP90 family includes the cytosolic HSP90alpha (HSP90a) and HSP90beta (HSP90(3) isoforms, as well as GRP94, which is found in the endoplasmic reticulum, and HSP 75/TRAP1, which is found in the mitochondrial matrix.

The term “HSP90 chaperone pathway” or “HSP90 pathway” refers to any process involving the biological activity of HSP90.

The term “inhibit” or other forms of the word such as “inhibiting” or “inhibition” means to hinder or restrain a particular characteristic. It is understood that this is typically in relation to some standard or expected value, i.e., it is relative, but that it is not always necessary for the standard or relative value to be referred to. “Inhibits” can also mean to hinder or restrain the synthesis, expression or function of the protein relative to a standard or control. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels. For example, “inhibits HSP90” means hindering, interfering with or restraining the activity of the HSP90 protein relative to a standard or a control. The term “HSP90 inhibitor”, or “HSP90i” refers to an agent that reduces, decreases, or inhibits the expression or activity of HSP90 or the HSP90 chaperone pathway.

“Treatment” or “treating” means to administer a composition to a subject or a system with an undesired condition (e.g., cancer). The condition can include one or more symptoms of the disease. “Prevention” or “preventing” means to administer a composition to a subject or a system at risk for the condition. The condition can be a predisposition to a disease. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur.

The term “dosing” or “dosage”, refers to the administration of a substance (e.g., an HSP90i) to achieve a therapeutic objective (e.g., the treatment of one or more symptoms of cancer, including a decrease in proliferation, metastasis or tumor volume). The terms “continuous dosing regimen”, “continuous dosing”, and “continuous administration”, as used herein, refer to the time course of administering the substance to a subject to achieve the therapeutic objective. The continuous dosing regimen relates to the presence of the drug within the plasma and, therefore, the efficacy of the drug to inhibit one or more of the functions of the target (e.g., HSP90).

The term “low dose” refers to a dosage that is lower than the recommended maximum dose (e.g., clinically determined Maximum Tolerated Dose; MTD), of an active agent (e.g., an HSP90i), for example, as established by a human clinical Phase trial or extrapolated from studies conducted in cell culture or animal studies that are predictive of results in humans. In some forms, a “low dose” refers to a drug concentration that is not inherently cytotoxic, does not induce a heat shock response (i.e. Hsp70 induction), and does not elicit immunosuppression.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, weight, etc.), the disease or disorder being treated, as well as the route of administration, the pharmacokinetics of the agent being administered and the pharmacodynamic effects of the active.

In this method, an “effective amount” is less than that which causes systemic toxicity. In this case, systemic toxicity refers to two main components: 1) toxicities associated with high dose HSP90 exposure including ocular, renal, gastrointestinal, and hepatic toxicity; and 2) immunosuppression as measured by decreased expression of antigen presentation genes in PBMCs, neutropenia, and/or reduced T-cell proliferation in peripheral tissues.

### II. Methods of Use

Methods of inhibiting the HSP90 chaperone and simultaneously stimulating the immune system in a subject with cancer are described. The methods include administering to the subject an effective amount of an HSP90i to reduce the activity of the HSP90 chaperone pathway in the subject compared to an untreated control. The amount of the HSP90i administered does not completely abrogate the function of HSP90 in the subject and does not induce systemic toxicity (i.e., a sub-toxic amount). This regimen contrasts with previous clinical testing of HSP90i, where the agent was administered with the goal of near complete or complete disruption of HSP90 function to kill cancer cells. In previous applications using HSP90i, activation of the heat shock response (i.e. Hsp70 induction) was used as a biomarker for HSP90i efficacy in cancer patients. In the methods described herein, activation of the heat shock response more than 10% of maximal induction is an indication that the dose of HSP90i is too high and should be scaled down to promote increased antigen presentation and prevent immunosuppression. In some embodiments, administration of the sub-toxic dose is repeated one or more times to maintain a continuous sub-toxic plasma concentration of HSP90i. The methods simultaneously stimulate the subject's immune system by administering to the subject one or more doses of one or more immunostimulatory molecules. Therefore, combination therapies for treating cancer in a subject in need thereof including reducing HSP90 function and simultaneously stimulating the immune system are provided.

A. Methods of Treating Cancer

It has been discovered that continuous, sub-toxic doses of inhibitors of HSP90 can sensitize cancer cells for killing by tumor-specific immune cells. It may be that the incomplete inhibition of HSP90 chaperone and HSP90-mediated chaperone pathway disrupts the HSP90 machinery sufficiently to enhance immune presentation of tumor antigen at the surface of cancer cells, while maintaining the basal function of HSP90 that is required for maintenance of cellular homeostasis. The mechanism of action is thought to involve the biological functions of immune effector cells that selectively kill cancer cells. Therefore, methods including co-administration of one or more agents that stimulate the immune system are also provided. The combination therapies and treatment regimens include administering to an individual with cancer an effective amount of a sub-toxic dose of a HSP90i and an immunostimulatory agent to treat the cancer or symptom thereof. In some embodiments, the one or more HSP90i(s) and one or more immunostimulatory agents are administered together, such as part of the same composition. In other embodiments, the one or more HSP90i(s) and one or more immunostimulatory agents are administered separately and independently at the same time or at different times (i.e., administration of the HSP90i(s) and immunostimulatory agent(s) is separated by a finite period of time from each other). Therefore, the term “combination” or “combined” is used to refer to either concomitant, simultaneous, or sequential administration of the HSP90 inhibitor(s) and immunostimulatory agent(s). The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

When used for treating cancer, the amount of HSP90 inhibitor(s) in a pharmaceutical dosage unit, or otherwise administered to a subject, can be the amount effective to treat one or more symptoms of the cancer, such as to reduce the proliferation, viability, or a combination thereof of the cancer cells when administered in combination with one or more immunostimulatory agent(s). Likewise, the amount of immunostimulatory agent(s) present in a pharmaceutical dosage unit, or otherwise administered to a subject can be the amount effective to reduce the proliferation, viability, or a combination thereof of the cancer cells when administered in combination with HSP90 inhibitor(s). Therefore, in some embodiments, the amount of the active agents is effective to reduce, slow or halt tumor progression, to reduce tumor burden, or a combination thereof. In some embodiments, the amount of the active agents is effective to alter a measureable biochemical or physiological marker. For example, if the cancer is prostate cancer, the amount of the active agents can be effective to reduce the level of prostate specific antigen (PSA) concentration in the blood compared to the PSA concentration prior to treatment.

In preferred embodiments, administration of the HSP90 inhibitor(s) and the immunostimulatory agent(s) achieves a result greater than when either agent is administered to the subject alone or in isolation (i.e., the result achieved by the combination is more than additive of the results achieved by the individual components alone). In some embodiments, the amount of one or both agents when used in the combination therapy is sub-therapeutic when used alone. The effect of the combination therapy, or individual agents thereof, can depend on the cancer to be treated, or progression thereof. For example, as illustrated in the Examples below, an HSP90i agent such as ganetespib (NCT01560416) can be used as a first or second line therapy for treatment of breast cancer. The Examples also illustrate that the effect of the combination therapies disclosed herein can be greater than the efficacy observed when either HSP90 inhibitor(s), or the immunostimulatory agent is used alone. Therefore, the combination therapy provides greater anti-cancer efficacy than the individual components when used alone. Accordingly, the combination provides enhanced response as compared to administration of the individual components alone. In other embodiments, the cancer killing effect of the combination is similar to the individual components, however the duration of efficacy of the treatment is longer because the cancer does not become resistant to the treatment. This allows the combination therapies to be administered in combination with, or as an alternative to, a first line therapy, or a second line or subsequent therapy.

1. Effective Amounts

As used herein the term “effective amount” or “therapeutically effective amount” means a dosage sufficient to treat, inhibit, or alleviate one or more symptoms of the disorder being treated or to otherwise provide a desired pharmacologic and/or physiologic effect.

Because the administration of the combination of low-doses of HSP90i, preferably in combination with one or more immunostimulatory agents, elicits an anti-tumor immune response, the amount of the combination administered can be expressed as the amount effective to achieve a desired anti-cancer effect in the recipient. For example, in some embodiments, the amount of the combination of agents is effective to inhibit the viability, metastasis, mass or proliferation of cancer cells in the recipient. In some embodiments, the amount of the combination of agents is effective to reduce the tumor burden in the recipient, or reduce the total number of cancer cells, and combinations thereof. In other embodiments, the amount of the combination of agents is effective to reduce one or more symptoms or signs of cancer in a cancer patient. Signs of cancer include cancer markers, such as, but not limited to, Prostate-Specific Membrane Antigen (PSMA) levels in the blood of a patient. In other embodiments, efficacy is assessed as a measure of the reduction in tumor volume and/or tumor mass at a specific time point (e.g., 1-5 days, weeks or months) following treatment.

a. Amounts of HSP90 Inhibitors

The amount of HSP90i administered to a subject with cancer in combination with an immunostimulatory agent is typically enough to reduce, decrease, or inhibit the HSP90 chaperone pathway in cancer cells of the subject, without giving rise to systemic toxicity in the subject (i.e., a “sub-toxic” dose of HSP90i). The term “systemic toxicity” refers to one or more adverse side-effects associated with HSP90 inhibition in non-cancer cells.

A sub-toxic dosage of an HSP90i is an amount that is less than the maximum tolerated dose (MTD) in humans, or less than the highest dose with acceptable toxicity (RP2D). RP2D is defined as the dose level producing around 20% of dose-limiting toxicity. A skilled physician will understand that the MTD and RP2D values vary for different HSP90i. MTD and RP2D can be expressed as an amount per unit body weight of the recipient (e.g., mg/kg), or as body surface-area based dosing (e.g., mg/m²). One skilled in the art will understand that conversion of unit amounts and dosages is routine in the art. For example, an amount of 10 mg per m² body size of the recipient correlates to a dosage of approximately 20 mg for an adult of 70 kg body weight and 200 cm tall (0.3 mg/kg), 20 mg per m² is a total dose of approximately 40 mg (0.6 mg/kg), 100 mg per m² is a total dose of approximately 200 mg (2.9 mg/kg), etc.

It has been established that the amount of an HSP90i that does not immuno-compromise the recipient is a “low-dose” that is between approximately 0.1% and 50% of the clinically-determined MTD or RP2D in humans for the HSP90i. In some embodiments, the amount of HSP90i is effective to produce a continuous plasma level in the recipient that is a fraction of that achieved by administration of the MTD or RP2D of the same HSP90i, for example, between approximately 0.1% and 50%. In some embodiments, the amount of HSP90i is effective to produce a blood plasma level that is between 1% and 50% of that achieved by administration of the MTD or RP2D dose. Therefore, the effective amount of an HSP90i can be 1%, 2%, 3%, 4%, 5%, 10%, 15%, or 20%, 25%, 30%, 35%, 40%, 45%, or 50%, of the clinically-determined MTD or RP2D dose (e.g., maximum recommended bolus dose) for the HSP90i. Typically, the amount is still below the dose that induces a systemic heat shock response as measured by HSP70 induction. In a preferred embodiment, the dose of an HSP90i for use in combination with one or more immunostimulatory agents is 5% of the clinically-determined MTD or RP2D for the HSP90i. This dosing gives rise to specific cancer cell killing by tumor-specific immune cells achieved by administering the amount that selectively inhibits HSP90 activity in cancer cells in the absence of systemic toxicity or reduced immune function in the recipient.

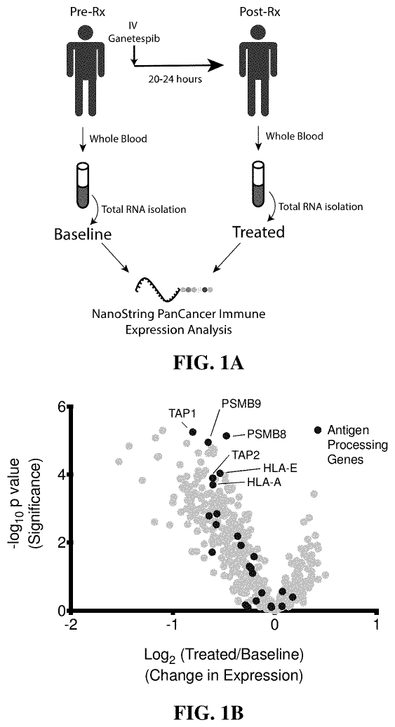

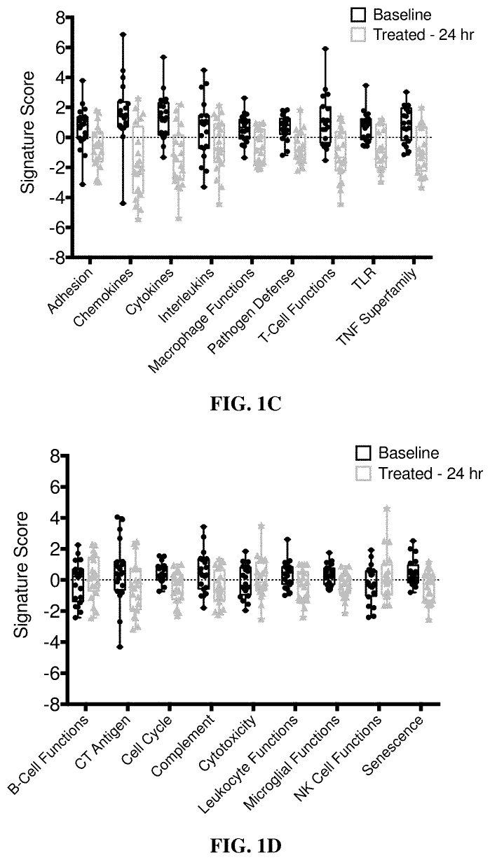

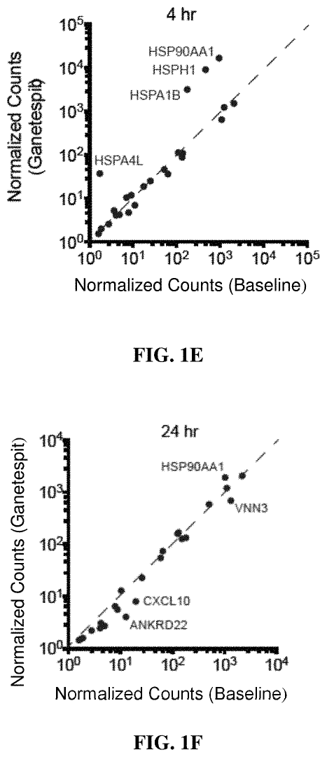

The examples below illustrate that high, intermittent doses of HSP90i reduced the expression of immune function genes relative to untreated controls (FIGS. 1B, 1C). However, reduced amounts of a related HSP90i resulted in the maintenance of immune-surveillance and increased immunity to tumor cells. Critically, the anti-tumor activity of low dose HSP90i was efficacious only in immunocompetent animals, suggesting a unique cell-non autonomous therapeutic effect resulting from continuous, low-dose administration of HSP90i. Therefore, in some embodiments HSP90i is administered in an amount effective to reduce or inhibit HSP90-mediated folding, activation, assembly, or function, or a combination thereof, whilst maintaining the normal function of the immune system in the recipient. Therefore, in some forms, the amount of an HSP90i is effective to induce or increase expression of genes associated with presentation of antigen in the context of MHC class I (i.e., expression of tumor antigen at the surface of cancer cells). Exemplary genes that can be enhanced by low-dose (sub-toxic) administration of HSP90i include immune-related genes Tap2, Tapbp, Psme1, Psme2, Psmb8, Psmb9, and Psmb10. In some embodiments, the amount of an HSP90i is effective to induce or increase expression of one or more genes including Tap2, Tapbp, Psme1, Psme2, Psmb8, Psmb9, and Psmb10 in the recipient. In some embodiments, the amount of an HSP90i is effective to increase the protein levels of one or more subunits of the immunoproteasome in the recipient. An exemplary immunoproteasome subunit that is up-regulated by the methods is the principal catalytic Psmb8 protein. Preferably, the amount of HSP90i administered is not sufficient to reduce or inhibit antigen presentation at the surface of cancer cells in the context of MHC Class I. For example, the amount of HSP90i does not reduce or inhibit the expression of proteins that enable immune surveillance, or the biological functions of the innate or adaptive immune system.

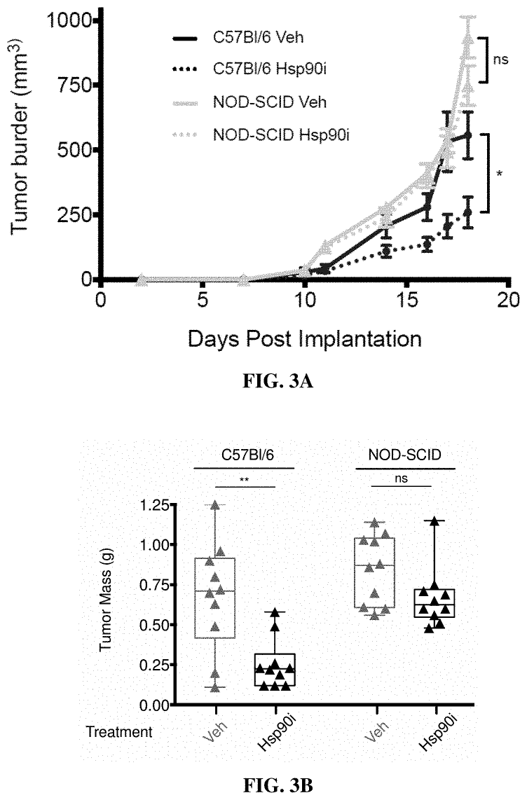

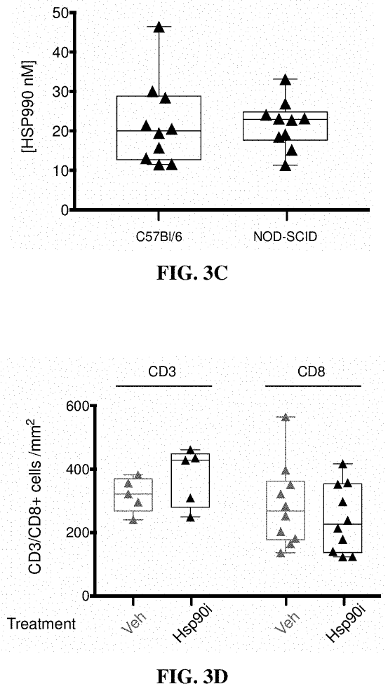

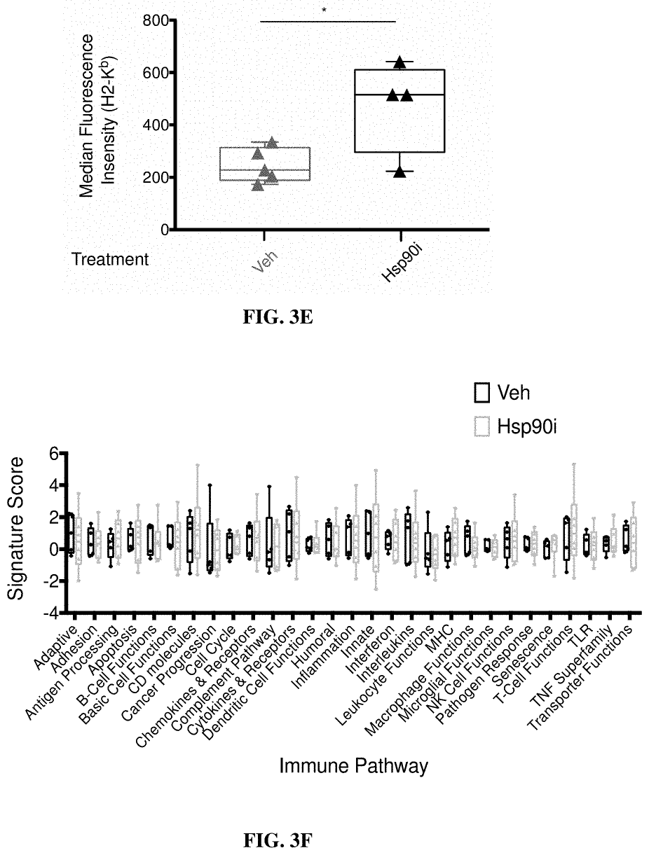

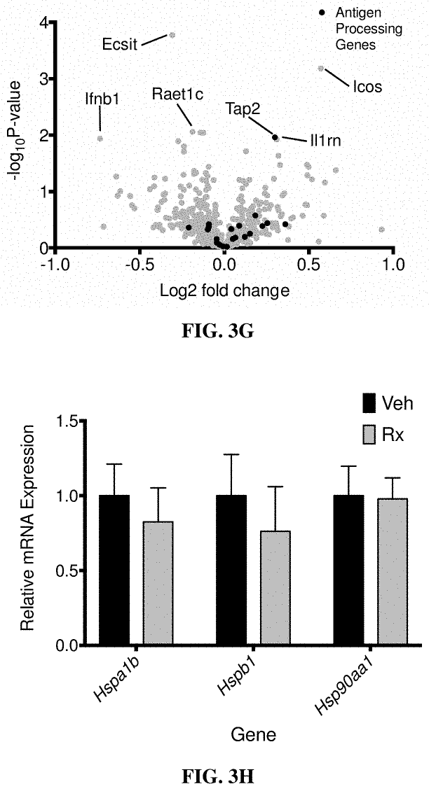

The total amount of HSP90i administered to the subject per dose as part of a combination therapy with an immunostimulatory agent can vary according to the route of administration. In some embodiments, the amount of HSP90i administered is between about 1 mg and 1,000 mg, for example, between 10 mg and 500 mg, between 20 mg and 100 mg. In an exemplary embodiment, orally bioavailable HSP90i is administered once or twice daily in an amount between 1 mg and 1,000 mg, inclusive. Typically, the amount of an HSP90i administered per single dose is effective to achieve a continuous (i.e., steady-state) plasma concentration that is ≤50% of that achieved by administering an amount that is the MTD or RP2D of the HSP90i. The amount administered in a single dose is effective to maintain drug concentrations below that which induce a systemic heat shock response as measured by induction of heat shock protein 70 kDa (HSP70). Phase I human clinical trial data established the pharmacokinetic profile of the orally bioavailable HSP90i NVP-HSP990. A single orally administered dose of 50 mg generated a peak plasma drug concentration of 1.31 μM, and was the recommended dose for subsequent Phase II human clinical trials. In contrast, a single oral dose of 2.5 mg resulted in a peak plasma concentration of 34 nM (Spreafico, et al., British Journal of Cancer, 112, pages 650-659 (2015)). Both doses exhibited drug half-lives between 20-25 hrs. In an exemplary embodiment, the HSP90i NVP-HSP990 is administered with daily oral dosing of 2.5 mg, to achieve a steady state drug concentration of 20-40 nM in plasma. This concentration is efficacious in in vivo preclinical studies (FIG. 3A-3J). In other embodiments, previously established pharmacokinetic parameters for distinct HSP90i's are used to guide similar dosing strategies effective to achieve continuous, low dose drug exposure.

b. Amounts of Immunostimulatory Agent

The amount of immunostimulatory agent administered to a subject with cancer in combination with an HSP90i is typically effective to induce, increase, enhance or stimulate immune responses in the recipient. Preferably, the amount of immunostimulatory agent administered does not give rise to undesirable immunological processes, such as auto-immune diseases and disorders. The term “undesirable immunological processes” refers to one or more adverse side-effects associated with dis-regulated immune activity in non-cancer cells. Depending on specific pharmacodynamic properties of the immunomodulatory treatment, administration of the immunomodulatory agent can begin at the same time as the course of HSP90i, or occur later in the course of HSP90i treatment. In some embodiments, the immunomodulatory agent would be administered intermittently throughout the course of continuous HSP90i treatment. In other embodiments, the immunomodulatory agent would be administered continuously, concurrent with HSP90i. In other embodiments, such as in the case of a cancer vaccine, the immunomodulatory agent would be given 1-3 times, prior to or at the beginning of HSP90i treatment, given with the intent to boost an antigen specific response against the tumor.

The immunostimulatory agent can be a non-specific stimulant of the immune system. Therefore, an effective amount of the immunostimulatory agent is an amount that induces or enhances one or more immunological processes in the recipient. In some embodiments, the amount of immunostimulatory agent elicits or enhances an anti-tumor immune response. The amount of immunostimulatory agent administered can be expressed as the amount effective to achieve a desired anti-cancer effect in the recipient. For example, in some embodiments, the amount of immunostimulatory agent is effective to inhibit the viability or proliferation of cancer cells in the recipient. In some embodiments, the amount of immunostimulatory agent is effective to reduce the tumor burden in the recipient, or reduce the total number of cancer cells, and combinations thereof. In other embodiments, the amount of immunostimulatory agent is effective to reduce one or more symptoms or signs of cancer in a cancer patient. Signs of cancer include cancer markers (e.g., PSA, PMSA, CEA).

Effective amounts of immunostimulatory agent per dose can vary according to the route of administration. In some embodiments, the amount of immunostimulatory agent administered is between about 1 mg and 1,000 mg, for example, between 10 mg and 500 mg, between 20 mg and 100 mg.

In an exemplary embodiment, an effective amount of an immunostimulatory agent is an anti-PD1 antibody administered via intravenous infiltration in an amount between 200 mg and 500 mg, inclusive.

2. Administration and Dosage Regimens

It has been established that continuous low-dose exposure to HSP90i elicits an anti-tumor immune response sufficient to reduce the viability of cancer cells in the recipient. Combinations of one or more HSP90i and one or more immunostimulatory agent can be administered by means appropriate for the combination of active agents. Exemplary routes of administration include enteral routes and parenteral routes. In some embodiments, the combination of HSP90i and immunostimulatory agent is administered as a single composition. In other embodiments, the combination of HSP90i and immunostimulatory agent is administered as part of a pharmaceutical composition that includes a pharmaceutically acceptable carrier.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated by the invention.

A treatment regimen of the combination therapy can include one or multiple administrations of the HSP90 inhibitor(s) and the immunostimulatory agent(s). A treatment regimen of the combination therapy can include multiple administrations of the HSP90 inhibitor(s), and one or multiple administrations of immunostimulatory agent(s).

In some embodiments, the immunostimulatory agent(s) is administered prior to the first administration of the HSP90 inhibitor(s). In other embodiments, the HSP90 inhibitor(s) is administered prior to the first administration of the immunostimulatory agent(s). For example, in some embodiments, the immunostimulatory agent(s) is administered at least 1, 2, 3, 5, 10, 12, 15, 20, 24 or 30 hours or days prior to or after administering of the HSP90 inhibitor(s). In other embodiments, the HSP90 inhibitor(s) is administered at least 1, 2, 3, 5, 10, 12, 15, 20, 24 or 30 hours or days prior to or after administering the immunostimulatory agent(s).

Dosage regimens or cycles of the agents can be completely, or partially overlapping, or can be sequential. For example, in some embodiments, all such administration(s) of the immunostimulatory agent(s) occur before or after administration of the HSP90 inhibitor(s). Alternatively, in other embodiments, administration of one or more doses of HSP90 inhibitor(s) can be temporally staggered with the administration of immunostimulatory agent(s) to form a uniform or non-uniform course of treatment whereby one or more doses of HSP90 inhibitor(s) are administered, followed by one or more doses of immunostimulatory agent(s), followed by one or more doses of HSP90 inhibitor(s); or one or more doses of immunostimulatory agent(s), are administered, followed by one or more doses of HSP90 inhibitor(s), followed by one or more doses of immunostimulatory agent(s); etc., all according to whatever schedule is selected or desired by the researcher or clinician administering the therapy. Diseases that can be treated using the described methods are discussed in more detail below.

In some embodiments, the HSP90i is administered in a regimen that is different from that with which the immunostimulatory agent is administered. For example, the first administration of active agents can include simultaneous dosing of both the immunostimulatory agent and the HSP90i, for example, as an admixture. The second or subsequent administrations of each active agent can be timed according to the desired blood-serum levels of each agent, for example, according to the serum half-life of each agent, respectively. The methods administer HSP90i in a regimen that provides a constant, low level of HSP90i effective to enhance anti-tumor immunity in the recipient. Therefore, in some embodiments, the HSP90 inhibitor and immunostimulatory agent are administered with sufficient frequency to maintain a blood-plasma level to induce an anti-tumor immune response. Co-administration of the immunostimulatory agent can be less frequent, or more frequent than the HSP90i. Therefore, in some embodiments, the methods administer the HSP90i more frequently than the immunostimulatory agent. In other embodiments, the methods administer the immunostimulatory agent more frequently than the HSP90i.

In some embodiments, a dose of one or more inhibitors of HSP90 is delivered to a subject as one or more doses to raise the blood concentration of the one or more inhibitors to a desired level. The dose can be given by any appropriate means, such as via injection or infiltration, or via oral ingestion. The repeating regimen of the dose can be varied depending upon the desired effect and the symptoms of the subject to be treated. Thus, the desired blood concentration of one or more HSP90 inhibitors can be maintained for a desired period of time. Typically, the blood-plasma level of HSP90i is maintained in the recipient at a level that is approximately 0.1%-50% that of the MTD throughout the duration of the treatment. The blood-plasma level of the immunostimulatory agent must be below that at which undesirable immunological side-effects, such as hypersensitivity, occur.

Treatment can be continued for an amount of time sufficient to achieve one or more desired therapeutic goals, for example, a reduction of the amount of cancer cells relative to the start of treatment, or complete absence of cancer cells in the recipient. Therefore, in some embodiments, the HSP90i is not administered on an intermittent basis. Treatment can be continued for a desired period of time, and the progression of treatment can be monitored using any means known for monitoring the progression of anti-cancer treatment in a patient. In some embodiments administration is carried out every day of treatment, or every week, or every fraction of a week. In some embodiments treatment regimens are carried out over the course of up to two, three, four or five days, weeks, or months, or for up to 6 months, or for more than 6 months, for example, up to one year, two years, three years, or up to five years.

Parenteral administration of the composition, if used, is generally characterized by injection. Injectable formulations can be prepared in conventional forms, either as liquid solutions or suspensions, solid forms suitable for solution of suspension in liquid prior to injection, or as emulsions. Administration involving use of a slow release or sustained release system, such that a constant dosage is maintained, is also discussed.

Pharmaceutical compositions including one or more inhibitors can be administered in a variety of manners. For example, the compositions can be administered intravenously (i.v.), intraperitoneally (i.p.), intramuscularly (i.m.), subcutaneously (s.c.), intracavity (i.c.), or by endotracheal (i.t.) delivery. In some embodiments, antibodies and antigen binding fragments thereof are delivered to a subject by endotracheal delivery. The compositions may be administered parenterally (e.g., infiltration), by injection, or by other means appropriate to a specific dosage form, e.g., including administration by inhalation of a lyophilized powder. The HSP90i and immunostimulatory agents can be administered as a single pharmaceutical composition including both agents, or c-administered to the same subject as two or more individual pharmaceutical compositions.

In some embodiments, when one or more HSP90 inhibitors is delivered separately from the immunostimulatory agent, the HSP90i is delivered by incorporating the inhibitor into a prosthesis, such as a medical device or tissue graft, for example, by loading the inhibitor(s) into or onto a structural or sealing material of the device. The rate of release of the inhibitor(s) may be controlled by a number of methods including varying one or more of the ratio of the absorbable material to the agent, the molecular weight of the absorbable material, the composition of the inhibitor(s), the composition of the absorbable material, the coating thickness, the number of coating layers and their relative thicknesses, the inhibitor concentration, and/or physical or chemical binding or linking of the inhibitor(s) to the device or sealing material. Top coats of polymers and other materials, including absorbable polymers, may also be applied to control the rate of release of the one or more inhibitors.

3. Cancers to be Treated

Cancer is a disease of genetic instability, allowing a cancer cell to acquire the eight hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell.; 144:646-674, (2011)).

The ATP-dependent chaperone HSP90 plays a pivotal role in the acquisition and maintenance of each of these capabilities. Therefore, inhibition of HSP90 leads to the degradation of these oncogenic clients and abrogates the six hallmarks of a cancer cell simultaneously (reviewed in Tatokoro, et al., EXCLI J. 14: 48-58 (2015)).

Malignant tumors which may be treated are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Methods for treating cancer in a subject in need thereof by administering to the patient an effective amount of one or more HSP90 inhibitor(s) in combination with an effective amount of one or more immunostimulatory agent(s), can treat carcinomas, sarcomas, lymphomas and leukemias. A non-limiting list of cancers that can be treated by the methods includes lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, skin cancer, liver cancer, melanoma, squamous cell carcinomas of the mouth, throat, larynx, and lung, colon cancer, cervical cancer, cervical carcinoma, epithelial cancer, renal cancer, genitourinary cancer, pulmonary cancer, esophageal carcinoma, head and neck carcinoma, large bowel cancer, hematopoietic cancers; testicular cancer; colon and rectal cancers, and pancreatic cancer.

The data in Example 7 demonstrates that HSP90i stimulates an anti-tumor immune response in tumors with a high mutational burden, when a sufficient/high amount of neoantigens is present. Cancers with a high mutational burden can be characterized based on the prevalence of somatic mutations in their genome and/or the presence of one or more markers. Methods for determining mutational burden are known in the art. For example, cancers exhibiting microsatellite instability or deficiency of one or more components involved in DNA repair (e.g., MSH2 have high mutational burdens, regardless of tissue of origin. Therefore, in some embodiments, the cancer to be treated has a high mutational burden. Such cancers include, but are not limited to, non-small cell lung cancer (NSCLC), lung squamous cell carcinoma (LUSC), bladder urothelial carcinoma (BLCA), colorectal carcinoma (CRC), head and neck squamous cell carcinoma (HNSC), uterine corpus endometrial carcinoma (UCEC), and glioblastoma multiforme (GBM).

B. Controls

The effect of a combination of HSP90i and immunostimulatory agent can be compared to a control. For example, in some embodiments, one or more of the pharmacological or physiological markers or pathways affected by combination of HSP90i and immunostimulatory agent treatment is compared to the same pharmacological or physiological marker or pathway in untreated control cells or untreated control subjects. In preferred embodiments, the untreated cells or the subject suffers the same disease or conditions as the treated cells or subject. For example, combination of HSP90i and immunostimulatory agent treated cells or subjects can be compared to cells or subjects treated with an HSP90 inhibitor alone. The cells or subjects treated with Hsp90 inhibitors alone can have a greater reduction in immunity expression, or a greater increase in pro-survival signaling than do cells or subjects treated with combination of HSP90i and immunostimulatory agent.

In some embodiments, the control can be the baseline level (e.g., levels before treatment) of a pharmacological or physiological marker in the subject to be treated. For example, in some embodiments, one or more of the pharmacological or physiological markers or pathways affected by combination of HSP90i and immunostimulatory agent treatment is compared to the same pharmacological or physiological marker or pathway prior to initiation of treatment.

In preferred embodiments, the methods deliver a combination of HSP90i(s) and immunostimulatory agent(s) in an amount effective to reduce, inhibit, or delay one or more symptoms of cancer in a subject.

### III. Compositions for Treating Cancer and Method of Making

Methods of making appropriate dosage formulations including providing dosage units for administration of HSP90i in a low level for daily administration, alone or in combination with an immunostimulatory agent. These may be provided in a single dosage unit or more typically in a kit, so that the HSP90i is administered daily and the immunostimulatory agent at less frequent intervals. The dosage units will typically be formulated as lyophilized or dry powders for resuspension for injection, or in a form for oral administration, although controlled release dosage forms may also be employed. In some embodiments, oral dosing with tablets twice or three times daily will achieve continuous, exposure to the effective concentration of HSP90 inhibitor. In other embodiments, extended release formulations in a tablet or capsule will be used to deliver the continuous exposure to drug. Finally, controlled release from implanted devices could also be used to deliver continuous exposure to drug.

Pharmaceutical compositions including a sub-toxic amount of one or more HSP90i effective to reduce the function of HSP90 pathway in a subject relative to an untreated control subject in combination with one or more immunostimulatory agents are provided. Compositions of HSP90i including benzoquinone ansamycin antibiotics can be formulated to include one or more immunostimulatory agents.

A. Inhibitors of Heat Shock Protein 90 (HSP90i)

Heat shock proteins (HSPs) are ATP-dependent chaperone proteins that become up-regulated in response to cellular environmental stresses, such as elevated temperature and oxygen or nutrient deprivation. HSP chaperones facilitate the proper folding and repair of other cellular proteins, referred to as “client proteins”, and also aid the refolding of misfolded proteins.

Of the several families of HSPs, the 90 kd “HSP90” family is one of the most abundant, representing approximately 1-2% of the total protein content in non-stressed cells and 4-6% of the protein content of cells that are stressed. The amino (N) terminal domain of HSP90 includes an ATP-binding site that is central to the chaperone function. The carboxyl (C) terminal domain of HSP90 mediates constitutive HSP90 dimerization. Conformational changes of HSP90 are orchestrated with the hydrolysis of ATP. HSP90 is highly conserved and facilitates the folding and maturation of over 200 client proteins, which are involved in a broad range of critical cellular pathways and processes. In non-stressed cells HSP90 participates in low affinity interactions to facilitate protein folding and maturation. In stressed cells HSP90 can assist the folding of dysregulated proteins, and is known to be involved in the development and maintenance of multiple diseases.

HSP90 maintains the conformation and stability of many oncogenic proteins, transcription factors, steroid receptors, metalloproteases and nitric oxide synthases that are essential for survival and proliferation of cancer cells (Whitesell, et al., Nature Reviews Cancer, 5, 761-772 (2005)). Thus, HSP90 client proteins have been associated with the development and progression of cancer. Furthermore, HSP90 is thought to contribute to maintenance of multiple neurodegenerative diseases that are associated with protein degradation and misfolding (proteinopathy), such as Alzheimer's disease, Huntingdon's disease and Parkinson's disease, through the misfolding or stabilization of aberrant (neurotoxic) client-proteins.

Inhibition of HSP90 function results in the misfolding of client proteins, which are subsequently ubiquitinated and degraded through proteasome-dependent pathways. Hence, inactivation of the HSP90 pathway represents a combinatorial attack on multiple signaling pathways and HSP90 inhibitors have been developed as therapeutics with efficacy in a broad variety of human cancers.

1. Benzoquinone Ansamycin Antibiotics

In some embodiments, the HSP90i is a benzoquinone ansamycin antibiotic. Exemplary benzoquinone ansamycin antibiotics include geldanamycin (GA; NSC 122750), 17-Allylamino-17-demethoxy-geldanamycin (17-AAG; NSC 330507; Tanespimycin), 17-dimethylaminoethylamino-17-demethoxy-geldanamycin (17-DMAG; NSC 707545; Alvespimycin), IPI-504 (Retaspimycin) (reviewed in Tatokoro, et al., EXCLI J. 14: 48-58 (2015)).

a. 17-AA G (Tanespimycin)

17-AAG was the first Hsp90 inhibitor to enter clinical trials. In vitro and in vivo, it has shown antitumor activity in various preclinical models, such as colon, breast, ovarian, and melanoma tumors. 17-AAG is not water-soluble and requires a diluent. Useful diluents include egg phospholipid and 4% DMSO. Side effects include hypersensitivity reactions, fatigue, nausea, vomiting, diarrhea, and transaminase elevations.

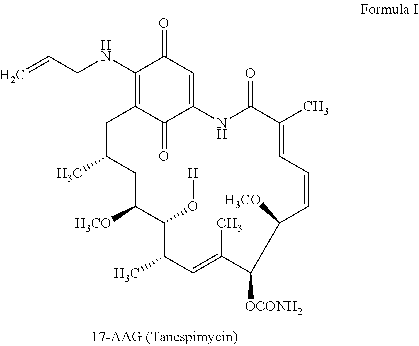

17-AAG is under phase I, II and III clinical trials for activity against Kidney tumors, non-Hodgkin's or Hodgkin's lymphomas, breast cancer, multiple myeloma, ovarian cancer, advanced solid tumors.

In one clinical trial, 17-AAG was administered intravenously (IV) over 60 minutes on days 1, 4, 8 and 11 of each 21 day cycle. The maximum tolerated dose (MTD) was identified as 150 mg/m². Hepatic toxicity and cardiac toxicity were dose limiting (Walker, et al., Leukemia & lymphoma 54(9),10(2013)).

b. 17-DMA G (Alvespimycin)

17-DMAG (Alvespimycin) was developed as a water-soluble analog of 17-AAG. Alvespimycin is associated with a longer plasma half-life, greater oral bioavailability, and less extensive metabolism.

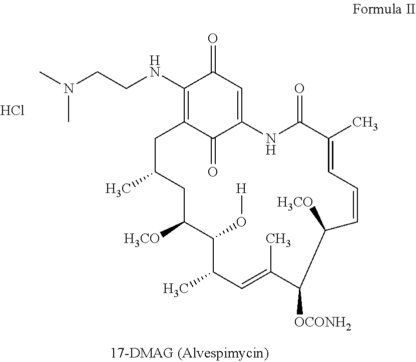

Oral and intravenous 17-DMAG (Alvespimycin) is under phase I clinical trials for activity against Melanoma, breast/prostate/ovarian cancers. Alvespimycin has an IC50 of 62 nM in a cell-free assay, and displays ˜2 times potency against human HSP90 as compared with 17-AAG. 17-DMAG treatment at 5 mg/kg or 25 mg/kg thrice per week significantly reduces tumor growth of TMK-1 xenografts in mice. 17-DMAG treatment at 25 mg/kg three times a week significantly suppresses tumor growth in mice. Administration of 17-DMAG at 10 mg/kg for 16 days significantly decreases the white blood cell count and prolongs the survival in a TCL1-SCID transplant mouse model.

In a phase one human clinical trial, patients with metastatic or unresectable solid tumors received alvespimycin hydrochloride via intravenous administration over 1 hour on days 1, 8, and 15. Courses were repeated every 28 days in the absence of disease progression or unacceptable toxicity.

c. IPI-504 (Retaspimycin)

IPI-504 is the hydroquinone hydrochloride salt of 17-allylamino-17-demethoxy-geldanamycin (17-AAG). IPI-504 has reached phase III clinical trials. IPI-504 demonstrates high aqueous solubility (>200 mg/mL). In vitro and in vivo IPI-504 interconverts with 17-AAG and exists in a pH and enzyme-mediated redox equilibrium due to oxidation of the hydroquinone (IPI-504) to the quinone (17-AAG) at physiological pH, and the reduction of 17-AAG by quinone reductases such as NQO1 to IPI-504.

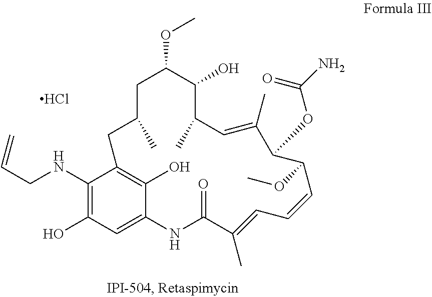 [SMILES: C=CCNc1c(O)cc2c(O)c1C[C@@H](C)C[C@H](OC)[C@H](O)[C@@H](C)/C=C(\C)[C@@H](OC(N)=O)[C@H](OC)/C=C\C=C(/C)C(=O)N2 ; Cl]

In a randomized, phase III trial of IPI-504 conducted in patients with metastatic and/or unresectable gastrointestinal stromal tumors (GIST), the trial was terminated early due to the occurrence of four on-treatment deaths in the IPI-504 arm. These deaths were considered drug-related and included renal failure, liver failure, metabolic acidosis, and cardiopulmonary arrest. In some phase II studies, including patients with non-small cell lung cancer (NSCLC) and HER2-positive breast cancer (IPI-504 had an acceptable safety profile, with infrequent transaminase elevations).

Based on a human phase Ib trial, the maximum tolerated dose (MTD) and recommended phase II dose (RP2D) was 450 mg/m² intravenous (iv) for retaspimycin in combination with docetaxel 75 mg/m² (iv) once every three weeks. Median number of cycles was three (range 1-11) (reviewed in Hendricks, et al., Expert opinion on investigational drugs, V26, (5), pp. 541-550 (2017)).

Retaspimycin was given as a monotherapy at 225 mg/m2 twice a week for two weeks followed by 10 days off therapy, one cycle was 21 days. MTD from phase 1 trial was indicated to be 225 mg/m² when administered as an intravenous infusion over 30 minutes by either peripheral or central venous access (reviewed in Hendricks, et al., Expert opinion on investigational drugs, V26, (5), pp. 541-550 (2017)).

2. Resorcinol Derivative HSP90 Inhibitors

In some embodiments, the HSP90i is a synthetic resorcinol derivative (RD) HSP90 inhibitor. Synthetic RD HSP90i typically bind the N-terminal ATPase site of HSP90 with higher affinity than the natural nucleotides and prevent the chaperone from cycling between its ADP- and ATP-bound conformations.

Exemplary resorcinol derivative HSP90i(s) include AUY922 (Novartis) AT13387 (Onalespib), KW-2478, and STA-9090 (ganetespib, Synta).

a. Ver-52296/Nvp AUY922 (Luminespib)

In some embodiments, the inhibitor of HSP90 is NVP-AUY922 (Luminespib). Luminespib is a potent small molecule HSP90i showing significant activity against breast cancer cells in cellular and in vivo settings.

Novartis evaluated intravenous luminespib (AUY922) in a number of Phase I and Phase II studies in patients with advanced solid tumors, lymphoma, chemotherapy-resistant metastatic pancreatic cancer, refractory GIST, NSCLC, HER2+breast cancer, ER+ hormone therapy refractory breast cancer, Stage IIIb/IV NSCLC, refractory solid tumors and haematological cancers.

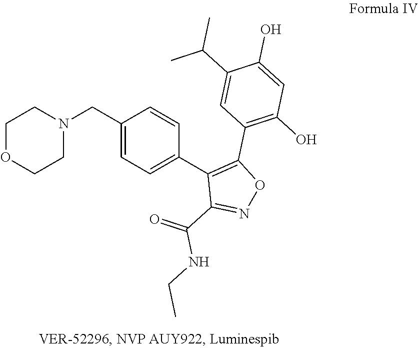 [SMILES: CCNC(=O)c1noc(-c2cc(C(C)C)c(O)cc2O)c1-c1ccc(CN2CCOCC2)cc1]

In a phase II clinical study, 70 mg/m² AUY922 was administered once weekly via intravenous infusion into a vein over about 60 minutes. The study treatment was given in 21 day cycles. Patients received an infusion of AUY922 on days 1, 8 and 15 of each cycle (once per week). In December 2009 Novartis announced that the maximum tolerated dose was 70 mg/m². Adverse effects of AUY922 have included diarrhea, nausea, fatigue, vomiting, and ocular toxicities (reviewed in Hendricks, et al., Expert opinion on investigational drugs, V26, (5), pp. 541-550 (2017)).

b. STA9090 (Ganetespib)

In some embodiments, the inhibitor of HSP90 is STA-9090 (Ganetespib). Ganetespib is a small molecule inhibitor of HSP90 that has favorable pharmacologic properties. Ganetespib is undergoing phase I, II and III clinical trials for treatment of patients with solid tumors, stage III/IV melanoma, HER2+ or triple negative breast cancer, stage IIIb/IV NSCLC, metastatic ocular melanoma, metastatic or unresectable GIST, advanced hepatocellular carcinoma, refractory metastatic colorectal cancer, AML, ALL and blast-phase CML, metastatic pancreatic cancer.

In a phase I trial, the MTD for Ganetespib as a monotherapy was determined as 216 mg/m², and the highest dose with acceptable toxicity, usually defined as the dose level producing around 20% of dose-limiting toxicity (RP2D) was 200 mg/m² when administered via intravenous infusion on days 1, 8 and 15, in a 4-weekly schedule. The most common side effects were diarrhea, fatigue, nausea, and anorexia, which have been manageable with standard care.

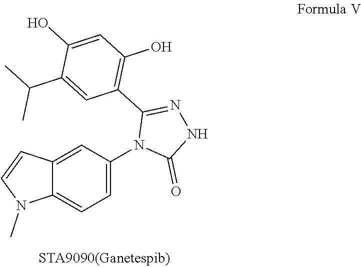 [SMILES: CC(C)c1cc(-c2n[nH]c(=O)n2-c2ccc3c(ccn3C)c2)c(O)cc1O]

In a phase II study, the MTD for Ganetespib as a combination therapy with crizotinib was determined as 200 mg/m2 on days 1 and 8 of a 21-day cycle. No ocular toxicity has been reported for STA-9090.

c. AT13387 (Onalespib)

In some embodiments, the inhibitor of HSP90 is AT13387 (Onalespib). Onalespib is small molecule inhibitor of HSP90 with IC50 of 18 nM in A375 cells, which displays a long duration of anti-tumor activity. The Kd for AT13387 binding is 0.7 nM. This compares to a Kd of 6.7 nM for the binding of the ansamycin (17-AAG) to the same site. The mean stoichiometry of binding for AT13387 is 1.03.

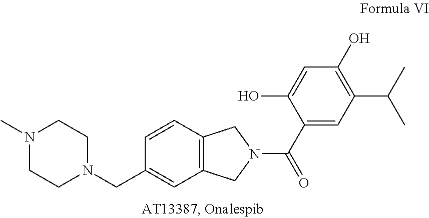 [SMILES: CC(C)c1cc(C(=O)N2Cc3ccc(CN4CCN(C)CC4)cc3C2)c(O)cc1O]

When given to test animals as a mono-therapy on an intermittent basis, AT13387 could be tolerated at doses of up to 70 mg/kg twice weekly, or 90 mg/kg once weekly.

Onalespib administered via oral or intravenous routes is currently undergoing phase Ib trials for treatment of patients with refractory solid tumors, e.g., HER2 negative breast cancer that has metastasized. MTD and R2PD for onalespib administered according to a dosing regimen of twice weekly for 3 weeks in a 4-week regimen was 120 mg/m2 (visual disturbances) and 260 mg/m2 (diarrhea, nausea, vomiting, fatigue, and systemic infusion reactions), respectively. For a regimen of six doses in a four-week schedule (dosing on day 1, 2, 8, 9, 15, 16) RP2D was 160 mg/m2 (liver enzyme abnormalities and gastrointestinal hemorrhage).

d. KW-2478

In some embodiments, the inhibitor of HSP90 is the investigational small molecule KW-2478.

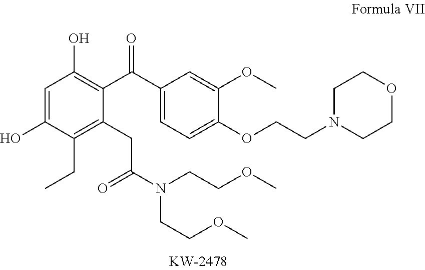 [SMILES: CCc1c(O)cc(O)c(C(=O)c2ccc(OCCN3CCOCC3)c(OC)c2)c1CC(=O)N(CCOC)CCOC]

KW-2478 has undergone phase I clinical testing in patients with refractory or relapsed multiple myeloma. Typically, KW-2478 is formulated to be administered via intravenous (iv) infusion. For Phase 1, the design was a standard 3+3 study of KW-2478 (130 or 175 mg/m²) and bortezomib (1.0 or 1.3 mg/m²) on Days 1, 4, 8, and 11 of a 21-day cycle utilizing four dose-escalation cohorts. For the Phase 2 portion of the study was designed to determine the preliminary efficacy of KW 2478 and bortezomib at the RP2D (KW-2478 175 mg/m2/bortezomib1.3 mg/m2).

e. CCT 018159

In some embodiments, the inhibitor of HSP90 is the CCT 018159. CCT 018159 was originally discovered by high-throughput screening at the Centre for Cancer Therapeutics, hence the CCT nomenclature.

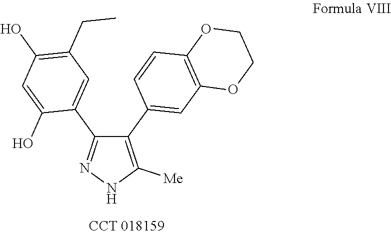 [SMILES: CCc1cc(-c2n[nH]c(C)c2-c2ccc3c(c2)OCCO3)c(O)cc1O]

3. Purine Scaffold HSP90i(s)

In some embodiments, the HSP90i is a purine, or purine-like analog. Synthetic purine analogues serve as HSP90i(s) with improved potency and physical/chemical properties. The unique structural features of the N-terminal nucleotide pocket as well as the shape adopted by ATP when HSP90-bound, have been used to rationally design a molecule to fit into this pocket. Major efforts have focused on probing the structure-activity relationship (SAR) of the aromatic moiety to the purine at C8-position, the nature of the linker between the PU-scaffold and the substituted aromatic ring, and the alkyl chain at N9 position.

Exemplary purine-like HSP90i(s) include Debi0932, PUH71, CNF-2024/BIIB021, MPC-3100 and BIIB021.

a. Debio 0932

In some embodiments, the inhibitor of HSP90 is the investigational molecule Debio 0932. Debio 0932 is an orally active HSP90i, with IC50s of 100 and 103 nM for HSP90α and HSP90β, respectively.

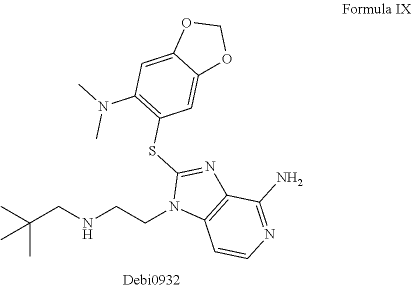 [SMILES: CN(C)c1cc2c(cc1Sc1nc3c(N)nccc3n1CCNCC(C)(C)C)OCO2]

Debio0932 is an investigational inhibitor of HSP90, which has undergone phase I clinical testing in patients with advanced solid tumors, lymphoma. In one clinical trial, Debio 0932 was administered as daily oral tablets at a starting dose of 250 mg four times per day (QD).

b. PUH71

In some embodiments, the inhibitor of HSP90 is the investigational molecule PUH71. PU-H71 is a potent and selective inhibitor of HSP90 with IC50 of 51 nM.

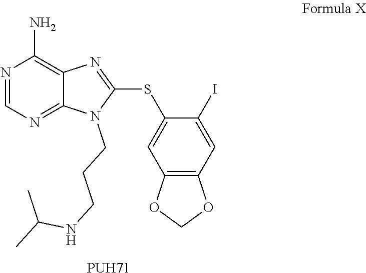

PUH71 is an investigational purine inhibitor of HSP90, which is undergoing phase Ib trials for treatment of patients with Refractory solid tumors, low-grade-non-Hodgkin's lymphoma, advanced metastatic solid tumor. PUH71 is administered via intravenous (iv) infusion. Clinical trials indicated MTD was in the range of 350-400 mg/m2.

c. CNF-2024/BIIB021

In some embodiments, the inhibitor of HSP90 is the investigational purine molecule CNF-2024/BIIB021. BIIB021 is an orally available, fully synthetic small-molecule inhibitor of HSP90 with Ki and EC50 of 1.7 nM and 38 nM, respectively.

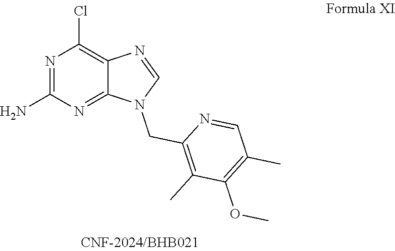 [SMILES: COc1c(C)cnc(Cn2cnc3c(Cl)nc(N)nc32)c1C]

CNF-2024/BIIB021 is an investigational purine inhibitor of HSP90, which is undergoing phase I and II trials for treatment of patients with refractory solid tumors, low-grade-non-Hodgkin's lymphoma, advanced metastatic solid tumor. CNF-2024/BIIB021 is administered orally. 800 mg was determined as the MTD for a twice-weekly oral dosing schedule.

d. MPC-3100

In some embodiments, the inhibitor of HSP90 is the investigational purine molecule MPC-3100.

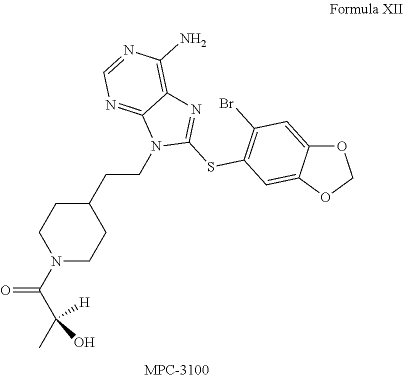 [SMILES: C[C@H](O)C(=O)N1CCC(CCn2c(Sc3cc4c(cc3Br)OCO4)nc3c(N)ncnc32)CC1]

MPC-3100 is an investigational purine inhibitor of HSP90, which is undergoing phase I trials for treatment of patients with relapsed or refractory cancer. MPC-3100 is administered orally.

4. Other HSP90i(s)

In some embodiments, the HSP90i is not a purine, or purine-like analog. Synthetic HSP90i typically bind the N-terminal ATPase site of HSP90 with higher affinity than the natural nucleotides and prevent the chaperone from cycling between its ADP- and ATP-bound conformations.

Exemplary small molecules and other inhibitors include TAS-116, Radicicol, Radanamycin, DS-2248, XL-888, NMS-E973, NVP-HSP990, Rifabutin, novobiocin, and SNX-5422.

a. TAS-116

In some embodiments, the inhibitor of HSP90 is TAS-116.

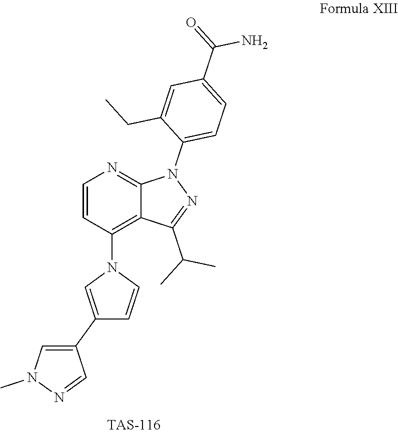 [SMILES: CCc1cc(C(N)=O)ccc1-n1nc(C(C)C)c2c(-n3ccc(-c4cnn(C)c4)c3)ccnc21]

TAS-116 is an investigational inhibitor of HSP90, which is undergoing phase I trials for treatment of patients with advanced solid tumors, HER2+ MBC, NSCLC harboring EGFR mutations (EGFRT790M+) or EGFR mutations (T790M−). TAS-116 is administered orally. Patients received 160 mg/day TAS-116 on a 5-days-on/2-days-off schedule.

b. Radicicol and Radanamycin

In some embodiments, the inhibitor of HSP90 is Radicicol (RD). RD lacks the toxic hydroquinone moiety of GA and its analogs, and is significantly less hepatotoxic than these analogs. Further, RD possesses nanomolar activity (IC50=20 nM) in cell lysates from Ras-transformed mouse fibroblasts, as well as purified human Hsp90 inhibition assays.

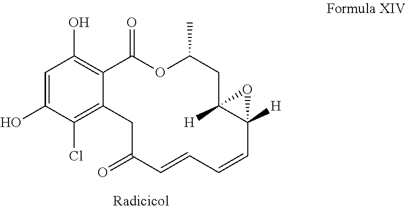

### Formula XIV: Radicicol

Radicicol is an investigational inhibitor of HSP90, which is undergoing phase I trials for treatment of patients with relapsed or refractory cancer. Radicicol is administered orally.

In some embodiments, the inhibitor of HSP90 Radanamycin (RDM). RDM a derivative of Radicicol.

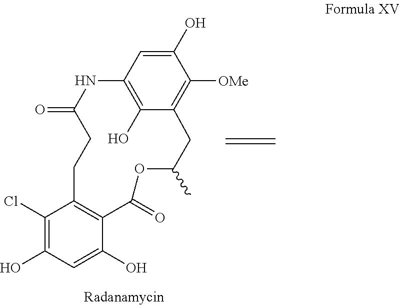 [SMILES: COc1c(O)cc2c(O)c1CC(C)OC(=O)c1c(O)cc(O)c(Cl)c1CCC(=O)N2]

### Formula XV: Radanamycin

c. DS-2248

In some embodiments, the inhibitor of HSP90 is the investigational molecule DS-2248. DS-2248 is undergoing phase I trials for treatment of patients with advanced solid tumors. DS-2248 is administered orally.

d. XL-888

In some embodiments, the inhibitor of HSP90 is the investigational molecule XL-888.

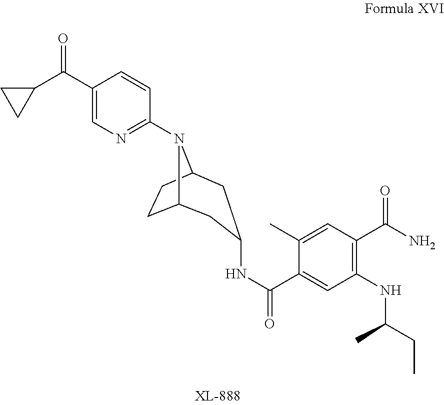 [SMILES: CC[C@@H](C)Nc1cc(C(=O)NC2CC3CCC(C2)N3c2ccc(C(=O)C3CC3)cn2)c(C)cc1C(N)=O]

XL888 (100 mg/kg) significantly induces the regression of, or growth inhibition (50%) of established M229R and 1205LuR xenografts in SCID mice. 15 days of XL888 treatment shows a robust (8.6-fold) increase in intratumoral HSP70 expression compared with controls.

XL-888 is undergoing phase I trials for treatment of patients with solid tumors, prostate cancer, unresectable BRAF mutant stage III/IV melanoma. To assess MTD XL-888 was administered orally, for example, 30-90 mg, twice weekly.

e. SNX-5422 and SNX-2112

In some embodiments, the inhibitor of HSP90 is the investigational molecule SNX-5422, which is the orally-available pro-drug of SNX-2112.

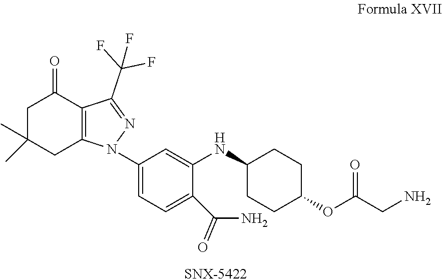

SNX-5422 is undergoing phase I trials for treatment of patients with refractory solid tumors, non-Hodgkin's lymphoma. SNX-5422 is administered orally. At doses of 42-100 mg/m2 of SNX-5422 taken every other day (qod), 2 of 3 patients (pts) with refractory neuroendocrine tumors (NET)s achieved stable disease for >8 cycles. The MTD of SNX-5422 was determined to be 75 mg/m² in pancreatic NETs and nonfunctional gastrointestinal and pulmonary NETs (Gutierrez, et al., Annals of Oncology, V27, (suppl. 6), vi136-vi148, (2016)).

f. NMS-E973

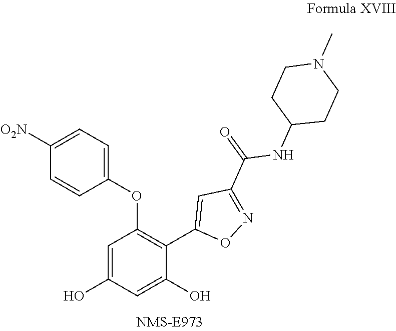 [SMILES: CN1CCC(NC(=O)c2cc(-c3c(O)cc(O)cc3Oc3ccc([N+](=O)[O-])cc3)on2)CC1]

In some embodiments, the inhibitor of HSP90 is the investigational molecule NMS-E973. NMS-E973 binds HSP90α with sub-nanomolar affinity and high selectivity towards kinases, as well as other ATPases.

NMS-E973 shows a favorable pharmacokinetic profile in test animals (administered 10 mg/kg i.v.) with selective retention in tumor tissue and ability to cross the blood-brain barrier. NMS-E973 (60 mg/kg i.v.) shows high antitumor efficacy in all the models tested, including A375 and A2780 xenografts.

g. NVP-HSP990.

In some embodiments, the inhibitor of HSP90 is NVP-HSP990. NVP-HSP990 (HSP990) is a potent and selective HSP90i for HSP90α/β with IC50 of 0.6 nM/0.8 nM. HSP90i is based on a 2-amino-4-methyl-7,8-dihydropyrido[4,3-d]pyrimidin-5(6H)-one scaffold, which is structurally distinct from other known HSP90i(s).

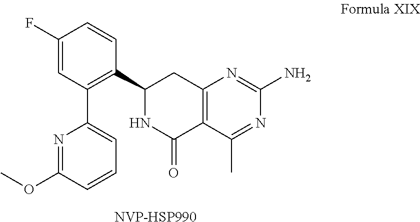

NVP-HSP990 is orally available. In one clinical trial, Heat-shock protein 990 was administered orally once or two times weekly on a 28-day cycle schedule in patients with advanced solid tumors. Fifty-three patients received HSP990 once weekly at 2.5, 5, 10, 20, 30, 50 or 60 mg, whereas 11 patients received HSP990 two times weekly at 25 mg. Median duration of exposure was 8 weeks (range 1-116 weeks) and 12 patients remained on treatment for >16 weeks. The single agent MTD/RP2D of HSP990 was declared at 50 mg once weekly.

Oral dosing of 2.5 mg NVP-HSP990 achieved a steady state drug concentration of 20-40 nM in plasma. Therefore, in some embodiments, an oral dose of 2.5 mg NVP-HSP990 is administered daily.

h. Rifabutin

In some embodiments, the inhibitor of HSP90 is the antibiotic Rifabutin. Rifabutin is an antibiotic; antitumor. Rifabutin interferes with HSP-90 molecular chaperone, enhances ubiquitination and protein degradation, and inactivates bacterial RNA polymerase.

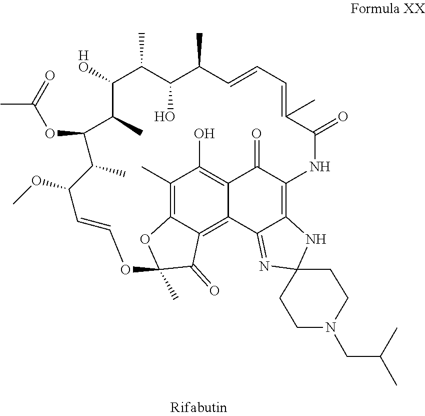 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(C)=O)[C@@H]1C)C4=O]

5. Inhibitors of the HSP90 Cycle

In some embodiments, the HSP90i is an agent that does not act by direct interaction with the HSP90 molecule itself, but acts to inhibit one or more of the down-stream molecules associated with the HSP90 “cycle” (i.e., “indirect HSP90i”). Hsp90, a protein that is highly conserved from prokaryotes to mammals, is known to associate with over 200 client proteins.

The HSP90 cycle includes (1) binding between the co-chaperone Hsp40 and a client protein; (2) recruitment of ATP-bound co-chaperone Hsp70; (3) ATP hydrolysis, which provokes a conformational change in Hsp70 and subsequent increased affinity for the substrate; (4) ADP-bound Hsp70 interacts with the Hop protein and Hsp90 dimers (formation of the “intermediate complex”); (5) ATP binds to Hsp90 dimers, inducing conformational changes and interaction with the co-chaperone p23; (6) dissociation from Hop; and (7) hydrolysis of ATP to ADP leads to disassembly of the complex and release of the mature client protein (reviewed in Scaltriti, et al., Clin Cancer Res; 18(17) (2012)).

Inhibitors that selectively target and inhibit or reduce one or more of the steps 1-7 of the above HSP90 cycle (i.e., an indirect HSP90i) are also described for use in combination with immunostimulatory agents to treat cancer.

It may be that silencing or otherwise preventing the expression or function the co-chaperones Hsp70, Hsp27, or HSF-1 increases sensitivity to Hsp90 inhibition. Therefore, in some embodiments, a direct HSP90i is combined with one or more indirect HSP90i, and one or more immunostimulatory agents.

B. Immunostimulatory Agents

Compositions for inhibiting cancer include an HSP90i and one or more immunostimulatory agents. Immunostimulatory agents initiate and enhance innate and adaptive immune processes, such as presentation of antigens, immune surveillance, T cell activation, and/or overcoming immunosuppression.

Compositions of immunostimulatory agents in an amount effective to increase or enhance the biological functions of the adaptive or innate immune system are described. Immunostimulatory agents manipulate the patient's immune system to stimulate, induce or enhance the biological functions of the patient's immune system to effectively recognize, attack and destroy cancer cells. Immunostimulatory properties include stimulation of antigenicity, adjuvant activity, and inflammatory responses.

Exemplary immunostimulatory agents include cytokines, ligands for immunostimulatory receptors, or antagonists for immunosuppressive receptors.

1. Pro-Inflammatory Molecules

Compositions of immunostimulatory agents include agents that induce a pro-inflammatory response.

T cells integrate the signals from the interface with antigen-presenting cells (the immunological synapse), and T cell activation only occurs when signals are able to overcome a certain threshold. Engagement of co-stimulatory molecules, such as CD28, decreases the amount of antigen necessary to elicit T cell activation. Inflammatory signals regulate expression of CD28 binding partners: B7-1 (CD80) and B7-2 (CD86).

In some embodiments, immunostimulatory agents include agents that increase serum levels of pro-inflammatory cytokines, including but not limited to, Interleukins such as IL-6, and IL-12, interferons, such as INF-γ, or tumor necrosis factors, such as TNF-α. In some embodiments, the immunostimulatory agents are cytokines. Cytokines activate immune cells, such as NK and CD8+ T cells, and can also inhibit tumor angiogenesis. Exemplary cytokines include one or more of IL-2, IL-4, IL-6, IL-7, IL-12, IL-15, IL-18, IL-19, IL-21, granulocyte-macrophage colony stimulating factor (GM-CSF), INF-γ, and TNF-α.

In certain embodiments, one immunostimulatory agent is T-cell growth cytokine, IL-15. IL-15 promotes the activation of a variety of immune cells, namely NK, NKT, and memory CD8+ T cells, and can overcome activation-induced cell death (AICD) caused by IL-2. In some embodiments, the immunostimulatory agents are co-stimulatory molecules, such as ICAM-1, ICAM-2, ICAM-3, B7-1, B7-2, CD40L, and combinations thereof

2. Adjuvants

Compositions of immunostimulatory agents include agents that are non-specific stimulants of the immune response. In some embodiments, one or more non-specific immunostimulants act as adjuvant for specific immunostimulation of anti-tumor immunity. Adjuvants act to accelerate, prolong, or enhance antigen-specific immune responses.

The immunostimulatory activity of DCs is capable of inducing secretion of inflammatory cytokines, such as TNF-α, IL-12, and IFN-γ, which are crucial for stimulation of T cells and recruitment of natural killer (NK) cells, as well as interleukin-1β (IL-1β), which promotes antibody production by B cells. APCs express a variety of cell-surface receptors that selectively bind to/recognize antigen, such as toll-like receptors (TLRs). Upon binding antigen, signaling via the APC receptors activate the APCs and stimulate an adaptive immune response. Substances that activate TLRs or other “danger” signal receptors are used in vaccines as adjuvants to stimulate an innate immune response through which a more effective adaptive immune response is generated. Agonists that stimulate TLRs are effective in functional maturation of DCs and their ability to prime T cells. The cationic polymer Polyethylenimine (PEI) has significant anti-tumor immune activity triggered by adjuvant activity due to activation of TLRs. Therefore, in some embodiments, the immunostimulatory agent is an adjuvant. Exemplary adjuvants include, but are not limited to, cationic polymers (e.g., PEI), anti-CD40 antibodies, Polyinosinic:polycytidylic acid (polyI:C), flagellin, aluminium phosphate, aluminium hydroxide, squalene, unmethylated, CpG oligonucleotide, prolactin, growth hormone, vitamin D, deoxycholic acid (DCA), imiquimod, resiquimod, gardiquimod, oil based adjuvants (e.g., MF59), purified plant extract QS-21, lipopolysaccharides, lipid A, heat stable antigen (HSA), and other TLR ligands.

3. Agents Enhancing Antigen Presentation

Immunostimulatory agents include agents that induce, stimulate or enhance presentation of antigen for immune surveillance by immune effector cells. For example, in some embodiments, the immunostimulatory agent is an antigen such as a tumor antigen. Presentation of tumor antigens by APCs activate tumor-specific cytotoxic T cells (CD8+ CTL).

a. Tumor Antigens

In some embodiments, immunostimulatory agents include tumor antigens. Compositions of tumor antigens are known in the art. Tumor antigens can be delivered to phagocytic cells, for example, to activate the cell to become an immunostimulatory antigen-presenting cell (APC). An exemplary antigen-presenting cell is a human mature dendritic cell (DCs). DCs expressing tumor antigens generate cytotoxic activity of tumor antigen-specific cytotoxic T lymphocytes (CTLs).

Exemplary tumor antigens include “melanoma antigen recognized by T cells 1” (MART-1; Melan-A), gp100, TRP-2, HER-2, MKI67 (antigen identified by monoclonal antibody Ki-67; the human protein that is encoded by the MKI67 gene), prostatic acid phosphatase (PAP), prostate-specific antigen (PSA), prostate-specific membrane antigen, early prostate cancer antigen, early prostate cancer antigen-2 (EPCA-2), BCL-2 (B-cell lymphoma 2; a protein encoded by the BCL2 gene), MAGE antigens such as CT7, MAGE-A3 and MAGE-A4, ERKS, G-protein coupled estrogen receptor 1, CA15-3, CA19-9, CA 72-4, CA-125, carcinoembryonic antigen, CD20, CD31, CD34, PTPRC (CD45), CD99, CD117, melanoma-associated antigen (TA-90), peripheral myelin protein 22 (PMP22), epithelial membrane proteins (EMP-1, -2, and -3), HMB-45 antigen, S100A1, S 100B, MUC-1, mucin antigens TF, Tn, STn, glycolipid globo H antigen.

Cross presentation of tumor antigens through MHC class I and class II on APCs to CD8+ or CD4+ T cells, respectively, stimulates T cells. CD8+ T cells undergo proliferation and differentiate into CTLs whereas CD4+ T cells differentiate into T-helper 1 (Th1) cells that can enhance anti-tumor CTL immune response at the tumor site.

4. Antagonists of Immuno-Suppressors

In some embodiments, the immunostimulatory agent is an agent that prevents, reduces or inhibits the biological activity of one or more immune-suppressor molecules. In some embodiments, the immunostimulatory agent directly or indirectly targets, reduces or prevents expression of an immunosuppressive cell surface receptor, such as PDL-1, CTLA-4, TGF-B, TIM-3, VISTA, LAG-3, IDO, KIR or IL-10 (reviewed in Kamphorst, et al., Vaccine 33(0 2): B21-B28 (2015); Dempke, et al., Eur J Cancer, V74, p. 55-72 (2017)).

Exemplary antagonists of immune-suppressors include antibodies, small molecules and functional RNAs (e.g., siRNA, miRNA). An exemplary immunostimulatory nucleic acid is silencing RNA (siRNA). Therefore, in some embodiments, the immunostimulatory nucleic acid is an siRNA or miRNA targeting an immunosuppressive receptor or molecule. In other embodiments, the immunostimulatory agent is an antibody, or antigen-binding fragment thereof. Typically, the antibodies are monoclonal antibodies, formulated for administration via intravenous (iv) infusion. An exemplary dosage for humans is between 1 and 15 mg/kg body weight of the recipient. An exemplary concentration for administration to humans is between 1 and 20 mg/ml.

In a preferred embodiment, the constant region of the monoclonal antibody is IgG1. Exemplary immunostimulatory monoclonal antibodies include anti-CTLA-4, and anti-PD1/PDL1.

a. Antagonists of CTLA-4

In some embodiments, the immunostimulatory agent directly or indirectly targets, reduces or prevents the function of CTLA-4. CTLA-4 is an inhibitory co-receptor that binds with higher affinity to B7 ligands than CD28. CTLA-4 is induced by TCR signaling, and it competes and physically excludes CD28 from the immunological synapse. In addition, CTLA-4 also recruits phosphatases that dephosphorylate key TCR/CD28 signaling molecules.

b. Antagonists of PD-1

In some embodiments, the immunostimulatory agent directly or indirectly targets, reduces or prevents the function of programmed death-1 receptor (PD-1)(CD279), and its ligand binding partners PD-L1 (B7-H1, CD274) and PD-L2 (B7-DC, CD273). PD-1 is related to CD28 and CTLA-4, but lacks the membrane proximal cysteine that allows homodimerization. PD-1 is an inhibitory receptor that modulates TCR and CD28 signaling through recruitment of the phosphatase SHP2. PD-1 binds PD-L1 (B7-H1/CD274) and PD-L2 (B7-DC/CD273). PD-L2 expression is restricted to antigen presenting cells (dendritic cells, monocytes and some B cell subsets), but PD-L1 expression is widespread. Expression of both PD-1 ligands is modulated by cytokines, such as IFN-γ. PD-L1 is expressed on hematopoietic and non-hematopoietic cells, including many tumor cells. Blocking interactions of PD-1 with PD-L1 can improve primary T cell responses. In some embodiments, the immunostimulatory agent is an anti-PD-L1 antibody. Anti-PD-L1 antibodies and antigen-binding fragments thereof are known in the art (see, for example, U.S. Pat. No. 9,624,298, which is hereby incorporated by reference in its entirety).

c. Antagonists of IL-10, VEGF or TGF

In some embodiments, the immunostimulatory agent directly or indirectly targets, reduces or prevents secretion of immunosuppressive cytokines, such as IL-10, and transforming growth factor beta (TGF-beta). Vascular endothelial growth factor (VEGF) is also associated with immunosuppression in the tumor environment. Therefore, in some embodiments, the immunostimulatory agent is an antagonist of VEGF.

5. Modulation of Regulatory T-cells (T-Reg)

In some embodiments, the immunostimulatory agent directly or indirectly targets, reduces or prevents the biological activities of regulatory T cell (T-Regs). Large numbers of T-reg cells are present at the tumor site, and increased effector T cell to T-reg cell ratio correlates with better prognosis.

Therefore, in some embodiments, the immunostimulatory agent directly or indirectly targets on or more receptors expressed at the surface of T-Reg cells, for example, to deplete the T-Reg cells. Exemplary markers that can be specifically targeted at the surface of T-Regs include CD25.

6. Co-stimulatory Antibodies

In some embodiments, the immunostimulatory agent is a co-stimulatory antibody. Co-stimulatory antibodies directly bind to and co-localize, cell-surface receptors that induce and support antigen-specific T cell activation. Exemplary molecules that are targeted and co-localized by co-stimulatory monoclonal antibodies include CD40, GITR, OX40, CD137, and ICOS.

Therefore, in some embodiments, the immunostimulatory agent directly or indirectly targets on or more receptors expressed at the surface of T-Reg cells, for example, to deplete the T-Reg cells. Exemplary markers that can be specifically targeted at the surface of T-Regs include CD25.

7. Combinations of Immunostimulatory Agents

In some embodiments, compositions for enhancing the antigen presentation, immune surveillance, T cell activation and killing of cancer cells include combinations or more than one class of immunostimulatory agents. Therefore, in some embodiments, Antagonists of Immuno-suppressors are combined with one or more pro-inflammatory molecules.

For example, in some embodiments, immunostimulatory agents include a PDL-1 antagonist in combination with IL-2.

Depletion of T-Reg cells can exhibit a most effective immunostimulatory effect when combined with anti-PD1 therapy. Therefore, in some embodiments, immunostimulatory agents include a PDL-1 antagonist in combination with a CD25 antagonist.

C. Additional Active Agents

Compositions of HSP90i(s) and immunostimulatory agents can include one or more additional active agents. Methods involving administration of a combination of HSP90i(s) and immunostimulatory agents can include administration of one or more additional active agents.

1. Additional Anticancer Agents

In some embodiments, compositions or combinations of HSP90i(s) and immunostimulatory agents include one or more additional agents that have anti-cancer activity in vivo. Exemplary additional anticancer agents include gefitinib, erlotinib, cis-platin, 5-fluorouracil, tegafur, raltitrexed, cytosine arabinoside, hydroxyurea, adriamycin, bleomycin, daunomycin, mitomycin-C, dactinomycin and mithramycin, vincristine, vinblastine, vindesine, vinorelbine, etoposide, teniposide, topotecan, camptothecin bortezomib anegrilide, tamoxifen, toremifene, raloxifene, droloxifene, iodoxyfene fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab (HERCEPTIN®), bevacizumab (AVASTIN®), gemtuzumab (MYLOTARG®), panitumumab (VECTIBIX®) or edrecolomab (PANOREX®), tyrosine kinase inhibitor, such as sorafenib (NEXAVAR®) or sunitinib (SUTENT®), cetuximab, dasatinib, imatinib, combretastatin, thalidomide, and/or lenalidomide, alkylating agents; alkyl sulfonates; aziridines, such as Thiotepa; ethyleneimines; anti-metabolites; folic acid-analogues, such as methotrexate (FARMITREXAT®, LANTAREL®, METEX®, MTX HEXAL®); purine analogues, such as azathioprine (AZAIPRIN®, AZAMEDAC®, IMUREK®, Zytrim®), cladribin (LEU-STATIN®), fludarabin phosphate (Fulda®), mercapto purine (MERCAP®, PURI-NETHOL®), pentostatin (NIPENT®), thioguanine (THIOGUANIN-WELLCOME®) or fludarabine; pyrimidine analogues, such as cytarabin (ALEXAN®, ARA-CELL®, UDICIL®), fluorouracil, 5-FU (EFUDIX®, FLUOROBLASTIN®, RIBOFLUOR®), gemcitabine (GEMZAR®), doxifluridine, azacitidine, carmofur, 6-azauridine, floxuridine; nitrogen-lost-derivatives, such as chlorambucil (LEUKERAN®), melphalan (ALKERAN®), chlornaphazine, estramustin, mechlorethamine; oxazaphosphorines, such as cyclophosphamide (CYCLO-CELL®, CYCLOSTIN®, ENDOXAN®), ifosfamide (HOLOXAN®, IFO-CELL®) or trofosfamide (IXOTEN®); nitrosureas, such as Bendamustine (RIBOMUSTIN®), Carmustine (CARMUBRIS®), Fotemustine (MUPHORAN®), Lomustine (CECENU®, LOMEBLASTIN®), chlorozotocine, ranimustine or nimustine (ACNU®); hydroxy-ureas (LITALIR®); taxens, such as docetaxel (TAXOTERE®), or paclitaxel (TAXOL®); platinum-compounds, such as cisplatin (PLATIBLASTIN®, PLATINEX®) or carboplatin (CARBOPLAT®, RIBOCARBO®); sulfonic acid esters, such as busulfan (MYLERAN®), piposulfan or treosulfan (OVASTAT®); anthracyclines, such as doxorubicin (ADRIBLASTIN®, DOXO-Cell®), daunorubicin (DAUNOBLASTIN®), epirubicin (FARMORUBICIN®), idarubicin (ZAVEDOS®), amsacrine (AMSIDYL®) or Mitoxantrone (NOVANTRON®); as well as derivates, tautomers and pharmaceutically active salts of the aforementioned compounds.

The present invention will be further understood by reference to the following non-limiting examples.

### EXAMPLES

### Example 1: HSP90i(s) Down-Regulate Expression of Diverse Immune-Related Genes in Cancer Patient at Clinically Recommended Doses

### Methods

Clinical Sample Collection

Blood samples for gene expression analyses were obtained with informed consent from patients participating in an IRB-approved clinical trial coordinated by the Dana-Farber Cancer Institute, Boston, Mass. (DFCI 11-477, NCT01560416). All samples were processed and analyzed by collaborating investigators in an anonymous fashion to preserve patient confidentiality.

Nanostring Analysis

Gene expression measurements using Nanostring codesets were performed with NanoString XT GEx kits. Analyses were performed on total RNA from clinical samples or mouse tumor tissue following manufacturer's instructions. Briefly, 100 ng total RNA (measured by Qubit (Invitrogen)), was mixed with Capture and Reporter probe sets and hybridized for 16-20 hours at 65° C. prior to ramping down to hold at 4° C. Hybridized samples were processed on a Nanostring Prep Station according to manufacturer's instructions and then scanned with an FOV setting of 1100. All files (.rcc) were analyzed using nSolver v3.0 software.

Statistical Analysis and graphics

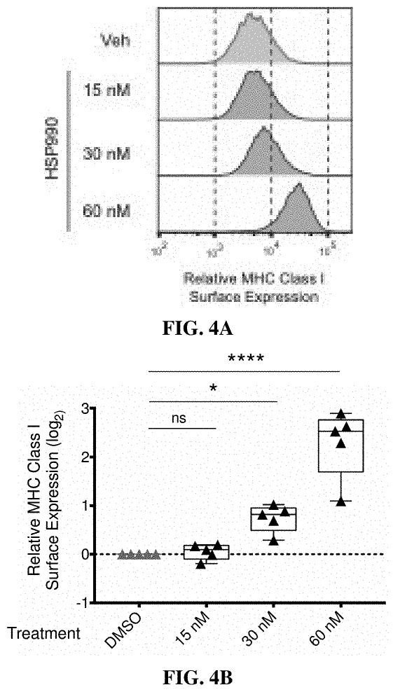

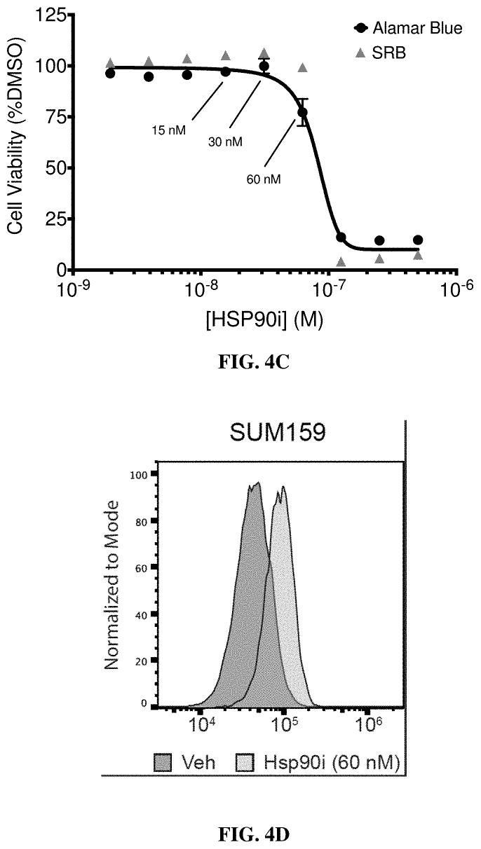

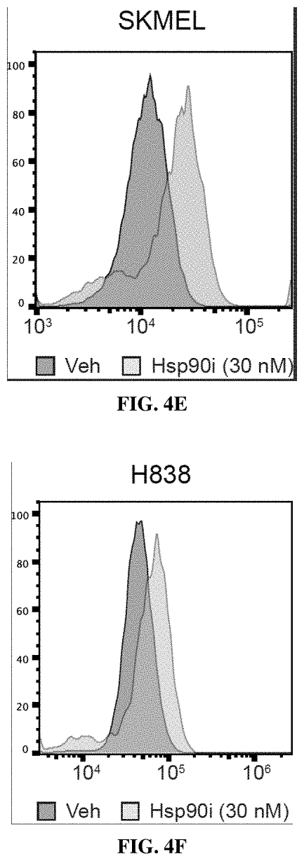

All graphical representations of data were generated in GraphPad Prism 7 or Adobe Illustrator or RStudio. NanoString data analysis was performed using nSolver 3.0 software and the Advanced Analysis module for PanCancer Immune Profiling. Differential expression analysis of RNAseq data was performed with the Bioconductor package DEseq2. For tumor growth curves, statistical analysis was performed with 3-Way ANOVA followed by Tukey's multiple comparison's test. Tumor mass comparisons were made with 2-Way ANOVA followed by Tukey's multiple comparison's test. MHC Class I staining of dissociated tumor cells was performed with two-tailed, unpaired, Welch's t-test. For qPCR, and FACS analysis comparisons, 2-WAY ANOVA followed by Tukey's multiple comparison's test was performed. Right-censored Kaplan-Meier analysis was used for the survival curves in FIG. 4c. * p<0.05, ** p<0.01, *** p<0.001, **** p<0.0001, ns=not significant.

### Results

As an approach to examine the impact of HSP90 compromise on the immune system in human cancer patients exposed to recommended dosing of an HSP90i, the RNA expression of immune-related genes in whole blood collected from 20 patients was profiled. These patients had metastatic estrogen-receptor positive breast cancer and were enrolled in a Phase II study investigating administration of the pure antiestrogen fulvestrant, with or without addition of the potent and selective HSP90i, ganetespib (NCT01560416).

Samples from ganetespib-treated patients were obtained at three time-points: prior to drug exposure, at 4 hr and at 20-24 hr post-intravenous administration of a heat-shock inducing dose of HSP90i (FIG. 1A). nCounter® Pan Cancer Immune Profiling Panels (XT-CSO-HIP1-12, NanoString Technologies Inc.) were used to simultaneously measure the relative expression of 730 immune-related genes and 40 housekeeping genes. Strikingly, in these clinical samples, broad down-regulation of expression across diverse immune-related genes induced by treatment with ganetespib at 20-24 hours post drug administration was observed (FIG. 1B). Among the most greatly affected genes were those involved in antigen presentation, a critical component of adaptive immunity and an essential step in effective recognition of tumors (Tscharke, D C et al., Nat Rev Immunol 15, 705-716, (2015); Gettinger, S. et al., Cancer Discov 7, 1420-1435, (2017)). The top six most significantly downregulated antigen-processing genes include TAP1, TAP2, PSMB9, PSMB8, HLA-E, and HLA-A (FIG. 1B). Genes involved in antigen production (PSMB8, PSMB9), antigen import into the endoplasmic reticulum (TAP1, and TAP2), and genes involved in surface presentation of antigen (HLA-A, HLA-E) were collectively downregulated following treatment (FIG. 1b). Using a pre-curated suite of gene-sets assembled to survey additional immune-related functions (Cesano, A. J Immunother Cancer 3, 42, (2015).), down-regulation of entire gene-sets at the expression level in response to administration of ganetespib was observed at its recommended Phase II dose (FIG. 1C, FIG. 1D, and FIG. 1E). Immune Pathway scoring of gene expression data using Nanostring Advanced Analysis was carried out. Signature scores were generated from a principal component analysis of gene-sets related to each immune pathway. 9 pathways shown are significantly downregulated following treatment (adjusted p<0.05) (FIG. 1C). Some of the immune-related pathways were not significantly affected following ganetispib treatment (FIG. 1D).

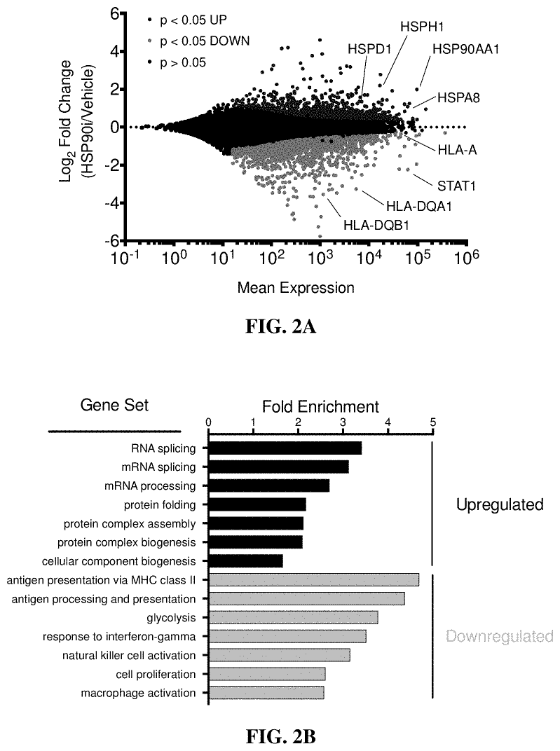

As an internal control, activation of the classical heat-shock response by HSP90i was also confirmed at four hours post drug administration. Expression of classical heat-shock genes of the clinical samples using a NanoString Codeset for heat-shock response genes was increased and returned to baseline levels by 24 hours (FIG. 1E and FIG. 1F). At the 4-hour time point, multiple heat shock protein genes are upregulated. By 20-24 hours, heat shock gene expression returned to baseline while immune related genes VNN3, CXCL10 and ANKRD22 exhibit reduced expression. Timing suggests that the downregulation of immune-related gene expression is downstream of the classical heat-shock response. To assess reproducibility, scatter plots are presented of the RNAseq data shown in FIG. 1C. Control and HSP90i conditions were analyzed in duplicate by RNAseq. Each point represents a single gene demonstrating high reproducibility (R2>0.98) across samples (FIG. 2G and FIG. 2H).

### Example 2: Ganetespib is Immunosuppressive in Ex Vivo Human Peripheral Blood Mononuclear Cells

### Methods

PBMC Isolation and RNA Sequencing

Whole blood was collected into lithium-heparin tubes and processed with Histopaque-1077 (Sigma) to isolate peripheral blood mononuclear cells per supplier's instructions. After overnight culture in RPMI160 with 20% autologous plasma, ganetespib (500 nM) or an equal volume of solvent vehicle (DMSO 0.1% v/v) was added to duplicate dishes. Incubation was continued for 24 hours after which cells were collected by centrifugation, lysed in RLT buffer (Qiagen) and total RNA isolated using an RNeasy kit per manufacturer's instructions (Qiagen). Sequencing library preparation was performed by the Whitehead Genome Technology Core using standard Illumina protocols and TruSeq adapters. Single-end, 40 bp sequencing was performed on a HiSeq 2000 instrument. Adapter sequences were trimmed, and reads were aligned to mm10 using TopHat2 and rpkm values generated with Cufflinks. Differential expression analysis was performed with the Bioconductor package DEseq.

### Data Accessibility

RNA-sequencing data has been deposited into the NCBI Gene Expression Omnibus (GSE113465). The mass spectrometry proteomics data have been deposited to the ProteomeXchange Consortium via the PRIDE partner repository with the dataset identifier PXD009542 and 10.6019/PXD009542.

### Results

To evaluate the immunosuppressive effect of high level HSP90 inhibition in a more controlled fashion, freshly isolated human peripheral blood mononuclear cells were cultured in ganetespib (500 nM for 20 hrs.) and the effects on RNA expression genome-wide were measured (FIG. 2A). HSP90 inhibition significantly altered the gene expression profile of these cells and Gene Ontology (GO) analysis revealed that that the most upregulated functional categories of genes were RNA splicing and protein folding, whereas the most downregulated categories were immune-related pathways (FIG. 2A and FIG. 2B).

Consistent with the clinical data, genes involved in antigen processing and presentation were significantly downregulated by treatment. Taken together with previous reports in preclinical models suggesting toxic HSP90 inhibition is immunosuppressive, these data suggest that current strategies for HSP90 inhibition in clinical trials result in broad compromise of innate and adaptive immune function (Graner, M W. Adv Cancer Res 129, 191-224, (2016); Graner, M W et al., Int J Hyperthermia 29, 380-389, (2013); Calderwood, S K et al., J Cell Biochem 113, 1096-1103, (2012); Bae, J. et al. J Immunol 178, 7730-7737 (2007); Bae, J. et al., J Immunol 190, 1360-1371, (2013)).

### Example 3: Anti-Tumor Response of Low Dose HSP90 Inhibition in Immunocompetent and Immunosuppressed Mice

### Methods

Mice

All experiments involving mice were performed under a protocol approved by the MIT Institutional Animal Care and Use Committee. MC38 cells growing in log phase were harvested by incubation with trypsin, washed 3× in PBS, and implanted (1×10⁵ cells/site) into the right inguinal region of 6-8 week old female C57B16 mice (Jackson Labs). Once palpable, tumors were monitored every other day via digital caliper measurements and volume calculated using the following formula: Length (mm)×Width (mm)×Width (mm)×520=tumor volume in mm³. Mice were euthanized by CO₂ inhalation, tumors were harvested and cut into thirds with a razor for histology, flow cytometry, and flash freezing in liquid N₂ for RNA/protein isolation.

For oral dosing with HSP90i, the average water consumption of the mice was calculated by measuring water bottle weight before and after 72 hours of housing to determine the average consumption per mouse, per day. Across all experiments, C57Cl/6 mice consumed approximately 4 mL every day. Using these water consumption values, and mouse weight, a 4 mg/mL stock solution of NVP-HSP990 (in 100% PEG400) was diluted directly into the drinking water to achieve a target dose of 0.5 mg/kg/day. HSP990 treatment was begun 72 hours prior to implantation of MC38 cells to allow the drug to reach steady state level prior to tumor challenge.

HSP990 Pharmacokinetics

Serum NVP-HSP990 levels were analyzed by sacrificing mice, performing a cardiac puncture, and isolating approximately 500 μL of whole blood. Blood was transferred to EDTA-coated microtubes and placed on ice. After all samples were collected, they were spun at 10,000×g for 15 min. The supernatant plasma was collected and stored at −80° C. until further processing. 2×10 μL aliquots of serum from each animal was extracted with 40 μL of ice cold acetonitrile and shaken for 30 min at 4° C. The acetonitrile solvent was spiked with 10 nM imatinib as an internal standard for mass spectrometry. Following extraction, samples were spun at 20,000×g for 15 min and the de-proteinated supernatant transferred to a clean Eppendorf tube prior to mass spectrometry analysis. Standard curves were prepared in plasma matrix at known concentrations of 0, 1.23, 3.7, 11.1, 33.3, 100, and 300 nM NVP-HSP990 and processed in parallel with the experimental samples.

Histology For histological sectioning, tumor tissue was fixed in 10% buffered formalin for 24 hours prior to transfer to 70% EtOH for storage until paraffin-embedding, sectioning and mounting on glass slides. For CD3 and CD8 staining, slides were dewaxed, and antigen retrieval was performed in citrate buffer (10 mM Citric acid, pH=6.0) and pressure-cooked. Slides were then cooled and washed in PBS+0.1% Tween-20 (PBST). Endogenous peroxidase activity was then blocked with Dako Dual Enzyme Block (Agilent Technologies) and endogenous proteins were blocked with 2.5% goat serum in PBST. Slides were then incubated with primary anti-CD8 antibody (1:400) overnight at 4° C. or anti-CD3 antibody (1:500) for 1 hr at 25° C. After incubation with primary antibody, slides were washed 3× with PBST, and then incubated with HRP-conjugated secondary antibody for 30 min at ° C. Slides were then washed 3× with PBST prior to development with DAB substrate for 3-5 min until positive staining was observed.

For CD8 and CD3 quantification, slides were scanned with a Leica High Resolution slide scanner and images imported into Aperio eSlide Manager. Two independent sections from each tumor were outlined and CD3/CD8+ cells were identified and quantified using the Nuclear-ID-v1 Algorithm. Individual tumor values are the average of the two tumor sections.

Flow Cytometry

For analysis of MHC Class I surface expression, cells were seeded into 12 well plates (125,000/well). 24-hours later, cells were treated with the indicated concentrations of HSP90i (NVP-HSP990) or DMSO vehicle. 72-hours after treatment, cells were dissociated with Accumax (Innovative Cell Technologies) and each treatment condition was distributed into 2 wells of a 96 well plate. Cells were spun at 500×g for 3 min, washed 1× in ice cold PBS supplemented with 2 mM EDTA and 0.5% FBS, and incubated with PE labeled anti-H2-Kb antibody (Clone AF6-88.5, Biolegend) or PE labeled isotype control (Clone MOPC-173, Biolegend) at a 1:400 dilution for 45 min on ice. After incubation with antibody, cells were washed 1× in ice cold PBS+10% FBS prior to analysis on a MACSQuant VYB flow cytometer. Cytometric data were plotted and analyzed with FloJo.

For analysis of MHC Class I on MC38 tumors, mice were sacrificed with by CO₂ inhalation, tumor tissue quickly dissected and placed on ice until processing by thorough mincing with a razor and filtration through a 70 μm cell strainer. Cells were then stained with H2-Kb antibody (1:400) or isotype control as described for cultured cells.

qRT-PCR

Total RNA isolation from cultured cells was performed after incubation in 6 well plates in the indicated conditions using RNeasy kits (Qiagen) according to the manufacturer's instructions and eluted in 50 μL MilliQ H2O. Eluted RNA was then treated with TURBO DNase (Ambion) and quantified by Nanodrop. 1.5 μg total RNA was then used for reverse transcription using the High Capacity cDNA Reverse Transcription Kit (Applied Biosystems) with random hexamers as primers. cDNA was then diluted 1:10 prior to PCR amplification with PowerUp SYBR Green Master Mix (Applied BioSystems) in a QuantStudio6 Real Time PCR System (Applied Biosystems) according to manufacturer's instructions. Ct values were normalized to Rp119 or Gapdh as housekeeping control and fold-changes calculated with the DMSO sample as reference.

For analysis of tissues, samples (2.5-5.0 mm³) were homogenized in 1 mL of Trizol (Molecular Research Center), phase separated with 200 μL chloroform, and precipitated with 100% isopropanol prior to resuspension in MilliQ H₂O and quantification with Nanodrop. RNA was then processed as described above for qRT-PCR.

### Results

The reservoir of HSP90 in cells is significantly higher than that needed to maintain essential HSP90-dependent functions under basal conditions (Taipale, M. et al., Nat Rev Mol Cell Biol 11, 515-528, (2010)). However, excess HSP90 is important for maintaining integrity of the cellular proteome in response to challenges such as hyperthermia, hypoxia, reactive oxygen species, and conformation-destabilizing mutations and polymorphisms. This role has given rise to the concept of HSP90 serving as a protein-folding “buffer”, and recent evidence suggests that impairing this buffer can have profound phenotypic consequences in diverse organisms without acute toxicity (Karras, G. I. et al., Cell 168, 856-866 e812, (2017); Queitsch, C et al., Nature 417, 618-624, (2002); Rutherford, S L. et al., Nature 396, 336-342, doi:10.1038/24550 (1998)). As many cancer cells express numerous mutant proteins that are highly dependent on HSP90 buffering capacity, continuous, low-dose administration of an HSP90i could alter the intracellular fate of these proteins and hence the malignant potential of cells expressing them without incurring the undesired systemic immunosuppressive effects of high-dose, overtly cytotoxic HSP90i exposures (Whitesell, L. et al., Proc Natl Acad Sci USA 111, 18297-18302, (2014).).

To test this hypothesis, a procedure to maintain continuous low nanomolar plasma concentrations of an orally bioavailable HSP90i (NVP-HSP990; herein referred to as Hsp90i) in mice was developed by administering it in their drinking water and a robust bioanalytical method to measure its concentration in plasma (FIG. 3A and FIG. 3B), confirming intact parent compound in the bioanalytical determination of plasma HSP90i levels. Recognizing the potential importance of effects on immune surveillance, the effect of continuous low dose treatment with Hsp90i was compared on the growth of transplanted murine MC38 tumors in syngeneic (C57Bl/6) and immunocompromised (NOD-SCID) hosts. In syngeneic hosts, continuous low dose administration of Hsp90i exerted a marked antitumor effect with a greater than 50% reduction in both tumor volume and tumor mass 18 days post implantation (FIG. 3C and FIG. 3D). Even more interestingly, HSP90i had no effect on tumor growth in NOD-SCID mice, which lack normal T- and B-cell function leading to profoundly impaired adaptive immune function (FIG. 3C and FIG. 3D). This outcome suggests that continuous low-dose exposure to HSP90i elicited an anti-tumor immune response sufficient to impede the progression of these very aggressive transplantable rodent tumors. Critically, the difference in HSP90i efficacy between C57Bl/6 and NOD-SCID mice could not be explained by differences in drug exposure because nearly identical plasma concentrations of HSP90i (˜20 nM) were present in both strains of mice (FIG. 3E). To pursue the mechanism(s) underlying the difference in the anti-tumor response of low dose HSP90 inhibition between immunocompetent and immunosuppressed mice, the immune infiltrate present in tumors from control and drug-treated mice was first characterized. Surprisingly, no significant difference was observed in the total number of CD3+ or CD8+ T-cells in the tumors, which suggests that the effect of HSP90i is not mediated through bulk T-cell recruitment (FIG. 3F). Given the expression profiling results demonstrating impairment by high-level HSP90i of antigen presentation in clinical samples and ex vivo treated human lymphocytes, the expression of MHC Class I on dissociated tumor cells was assessed. Intriguingly, higher levels of MHC Class I expression on the surface of dissociated MC38 tumor cells isolated from HSP90i treated mice were observed (FIG. 3G), suggesting that low dose HSP90i, rather than impairing, might actually stimulate antigen presentation on tumor cells and lead to more robust tumor recognition by immune cells.

To support this hypothesis, Nanostring expression analysis of MC38 tumor tissue revealed that low dose Hsp90i exposure did not result in broad downregulation in immune gene expression (FIG. 3H). This is in contrast to the profiling of human patients receiving conventional high-dose HSP90i therapy (FIG. 1B and FIG. 1C). Ecsit, Ifnb1, Raet1C, Tap2, Il1rn, and Icos are the top 6 genes most significantly altered in expression (FIG. 3I). No evidence of heat-shock activation by the continuous low-dose HSP90i administration protocol was observed (FIG. 3J). Low-dose HSP90i treatment of mice does not induce a heat-shock response in their tumor burdens.

### Example 4: Hsp90i Alters the Antigenic Profile of MC38 Cells

### Methods

### Cell Culture

MC38 cells were kindly provided by A. Sharpe from Dana Farber Cancer Institute. Cells were maintained in DMEM supplemented with 10% FBS and penicillin/streptomycin. SUM159, H838, SKMEL, and Colo cells were maintained in RPMI1640 supplemented with 10% FBS and penicillin/streptomycin. Cells were confirmed negative for mycoplasma contamination by PCR-based assay. For cell viability assays, 20,000 MC38 cells were seeded in to 96 well plates, allowed to attach for 24 hours, and then treated with serial dilutions of HSP90i (NVP-HSP990). Relative cell content per well was assayed after 72 hours treatment using alamar blue (R&D Systems) or Sulphorhodamine B (Sigma). For alamar blue assays, the stock solution was diluted 1:4 in PBS and then added directly to the 96 well plate at a 1:5 dilution (1:20 dilution final) and incubated at 37° C. under 5% CO₂ for 3 hours. Fluorescence as a measure of relative dye reduction was measured on an Envision plate reader (Perkin Elmer) with excitation at 544 nm and emission readings at 570 nm.

Flow Cytometry

For analysis of MHC Class I surface expression, cells were seeded into 12 well plates (125,000/well). 24-hours later, cells were treated with the indicated concentrations of HSP90i (NVP-HSP990) or DMSO vehicle. 72-hours after treatment, cells were dissociated with Accumax (Innovative Cell Technologies) and each treatment condition was distributed into 2 wells of a 96 well plate. Cells were spun at 500×g for 3 min, washed 1× in ice cold PBS supplemented with 2 mM EDTA and 0.5% FBS, and incubated with PE labeled anti-H2-Kb antibody (Clone AF6-88.5, Biolegend) or PE labeled isotype control (Clone MOPC-173, Biolegend) at a 1:400 dilution for 45 min on ice. After incubation with antibody, cells were washed 1× in ice cold PBS+10% FBS prior to analysis on a MACSQuant VYB flow cytometer. Cytometric data were plotted and analyzed with FloJo.

For analysis of MHC Class I on MC38 tumors, mice were sacrificed with by CO2 inhalation, tumor tissue quickly dissected and placed on ice until processing by thorough mincing with a razor and filtration through a 70 μm cell strainer. Cells were then stained with H2-Kb antibody (1:400) or isotype control as described for cultured cells.

Immunoblotting

Protein extraction was performed by washing cells 2× with PBS. A volume of lysis buffer (50 mM Tris pH 7.5, 150 mM NaCl, 1 mM EDTA, 1% Triton-X100, 0.1% SDS, 1× Halt Protease/Phosphatase inhibitor) equal to 1/10 the culture media volume was added directly to the culture dish and incubated for 2-3 min on ice. Lysates were then thoroughly scraped and pipetted into 1.5 mL tubes and incubated for an additional 30 min on ice. Lysates were then cleared by centrifugation at 14,000×g for 15 min and quantified using BCA Assay. Lysates were separated by SDS-PAGE and transferred to 0.2 μm nitrocellulose membranes prior to washing with PBST, blocking with 5% milk in PBST, and incubation with primary antibody overnight at 4° C. All primary antibodies were diluted 1:1000 in 2.5% milk in PBST. Membranes were then washed 4×5 min with PBST and incubated with HRP conjugated secondary antibody at a 1:10,000 dilution in 2.5% milk in PBST, and washed again 4×5 min with PBST. Detection of secondary antibody was performed with SuperSignal West Pico or Femto ECL reagents (Pierce) and digitally imaged on a ChemiDoc (BioRad).

MHC Class I Peptide Profiling

MC38 cells were cultured as described above. 10-10 cm (˜1×10⁸ cells) plates of MC38 cells were cultured for 72 hours in 0.1% DMSO or 50 nM HSP990. At the time of harvest, plates were washed 1× in PBS, and then cells were lifted using PBS+2 mM EDTA. Cells were then pelleted at 500 g for 5 min and the pellets washed twice more with ice cold PBS. Pellets were then resuspended in 1 mL IP lysis buffer (20 nM Tris pH 7.5, 150 mM NaCl, 1% CHAPS, 0.2 mM PMSF, 1× HALT protease/phosphatase inhibitors (Pierce). Lysates were then sonicated with a microtip sonicator for 3×10 s pulses to further disrupt cell membranes, and then centrifuged at 14 k g for 10 min. The supernatant was then quantified at 10 mg total protein lysate was used for the MHC Class IIP.

Peptide MHC isolation was performed using a modified immunoprecipitation and protein filtration protocol, as previously described. Briefly, for each sample, 250m of anti-H2-Kb clone (Y3, BioXCell) and 250 μg anti-H2Db (clone B22-249, Thermo-Fisher) were bound to 20 μL (bed volume) FastFlow Protein A Sepharose beads (GE Healthcare) by incubating for 3 hrs at 4° C. Beads were then washed with lysis buffer and 10 mg of lysate was added and incubated overnight rotating at 4° C. Beads were then centrifuged at 2000 rpm, washed twice with 1×TBS, and eluted with 10% acetic acid at room temperature. Eluate was then filtered using 10 kDa MWCO spin filters (PALL Life Science), which were passivated according to manufacturer's instructions and acidified prior to filtration. Isolated peptides were concentrated with vacuum centrifugation and stores at −80° C. until LC MS/MS analysis.

Mass Spectrometry Analysis of MHC Peptides

For MS analysis, peptides were resuspended in 0.1% acetic acid and loaded on a precolumn packed in house [100 μm ID×10 cm packed with 10 μm C18 beads (YMC gel, ODS-A, 12 nm, S-10 μm, AA12S11)]. The precolumn was then washed with 0.1% acetic acid and connected in series to an analytical capillary column with an integrated electrospray tip (˜1 μm orifice) with 5 uM C18 beads, prepared in house ([50 μm ID×12 cm with 5 μm C18 beads (YMC gel, ODS-AQ, 12 nm, S-5 μm, AQ12S05)].

Peptides were eluted using a 130 minute gradient with 10-45% buffer B (70% Acetonitrile, 0.2M acetic acid) from 5-100 minutes and 45-55% from 100-120 minutes at a flow rate of 0.2 mL/min for a flow split of approximately 10,000:1. Peptides were analyzed using a Thermo Q Exactive HF-X Hybrid Quadrupole-Orbitrap mass spectrometer. Standard mass spectrometry parameters were as follows: spray voltage, 2.5 kV; no sheath or auxiliary gas flow; heated capillary temperature, 250° C. The HF-X was operated in data-dependent acquisition mode. Full-scan mass spectrometry spectra [mass/charge ratio (m/z), 350 to 2,000; resolution, 60,000] were detected in the Orbitrap analyzer after accumulation of ions at 3e6 target value. For every full scan, the 15 most intense ions were isolated (isolation width of 0.4 m/z) and fragmented [collision energy (CE): 28%] by higher energy collisional dissociation (HCD) with a maximum injection time of 350 milliseconds and 30,000 resolution. Dynamic exclusion was set to 15 second.

Mass Spectrometry Data Analysis

Raw mass spectral data files were analyzed by first loading into Proteome Discoverer version 2.2 (Thermo Fisher Scientific) and searched against the mouse SwissProt database and mutant peptide MC38 database using Mascot version 2.4 (Matrix Science). Spectra were matched with a mass tolerance of 10 ppm for precursor masses and 20 mmu for fragment ions. Peptides were filtered according to an ion score ≥20, isolation interference≤30%, rank 1, and between 6-13 amino acids in length. Peptides from replicates 1, 3, and 5 were subjected to label free quantification using the minora feature detector in Proteome Discoverer with area as the quantification metric. Peptides that received quantification values in at least 2 matched replicate samples were used, and the fold change of treated over DMSO control was averaged across replicates.

### Results

To better understand the mechanism(s) by which HSP90i stimulates a more effective anti-tumor immune response to MC38 cells, experiments in cell culture were carried out. It was observed that HSP90i stimulated MHC Class I surface expression at concentrations well below the IC50 for cytotoxicity (FIG. 4A, FIG. 4B, and FIG. 4C). It was also found that HSP90i induced upregulation of MHC Class I expression not just on mouse MC38 cells, but also on human tumor cell lines originating from breast, melanocytes and lung (FIG. 4D, FIG. 4E, and FIG. 4F), which suggests that this HSP90i strategy may be broadly applicable.

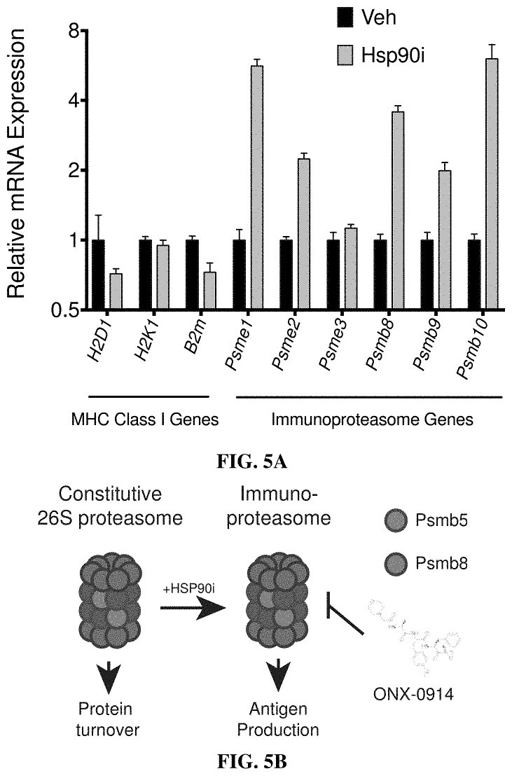

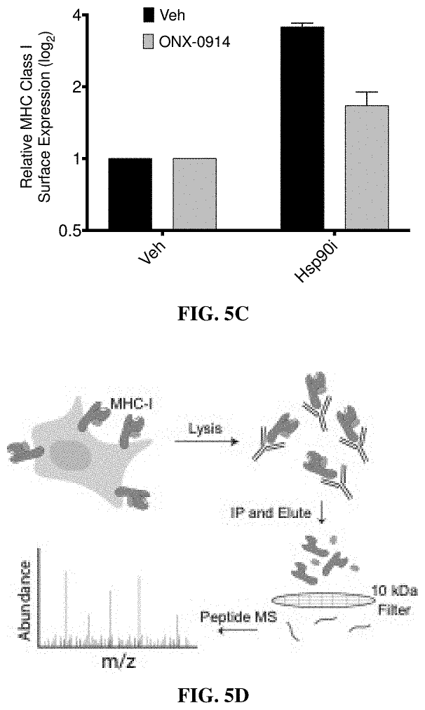

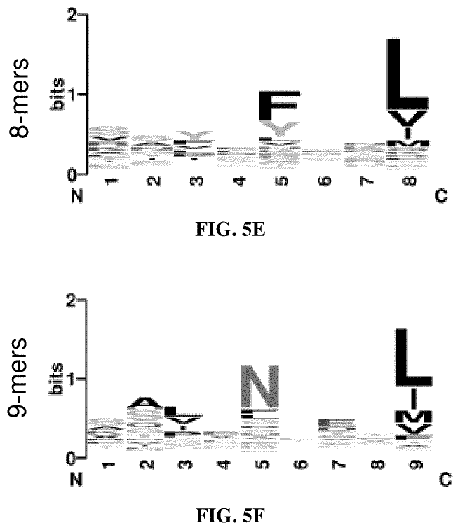

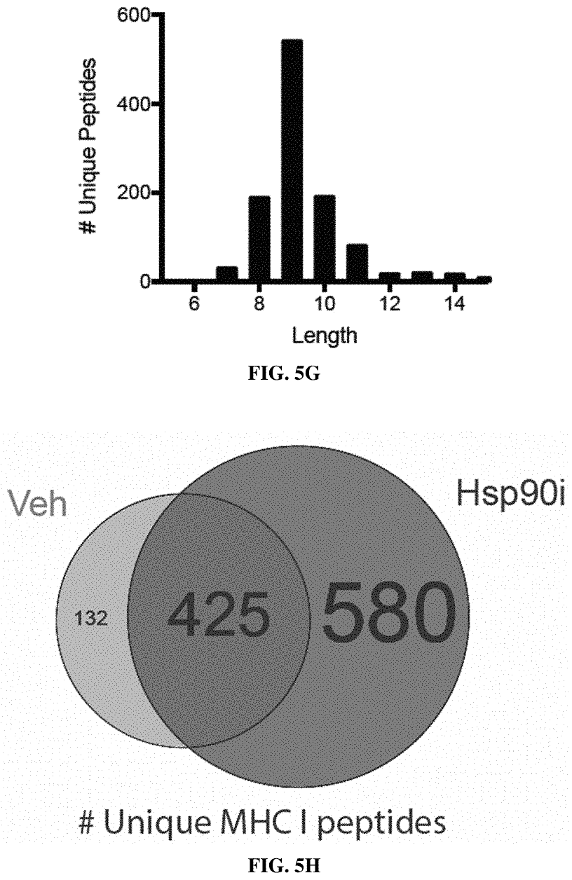

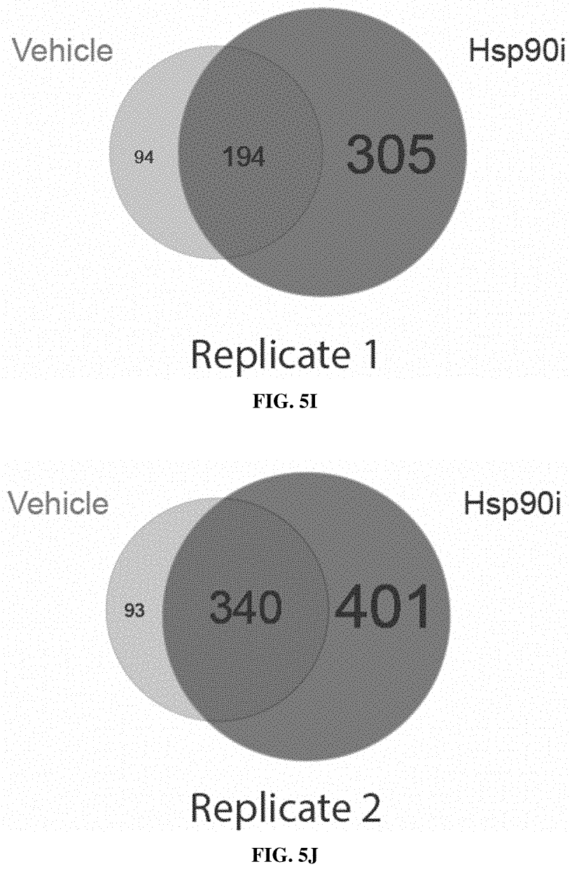

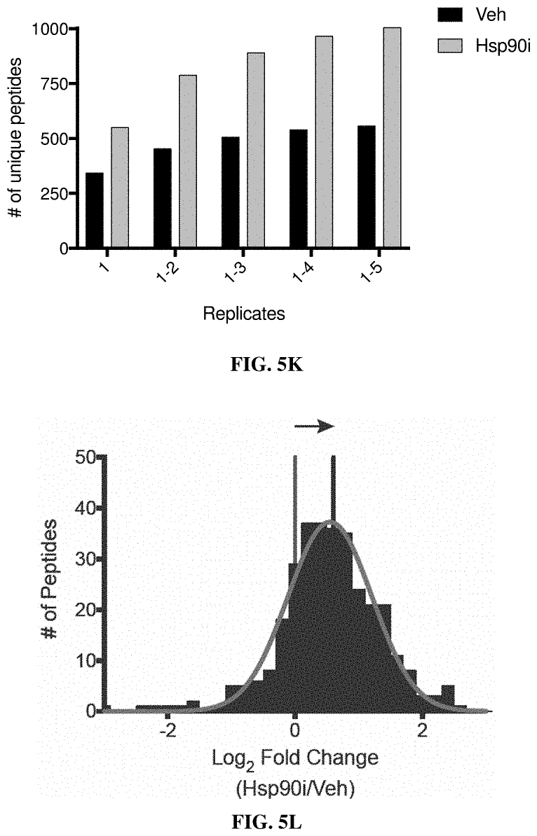

In contrast to canonical IFNγ-stimulated antigen presentation, exposure to sub-toxic HSP90i did not alter transcript levels of MHC Class I heavy chains H2-K1 and H2-D1 or the light chain beta-2-microglobulin (B2m), which indicates a distinct mode of action (FIG. 5A). Given the central role of the immunoproteasome in antigen presentation (Vigneron, N. et al., Curr Opin Immunol 24, 84-91, (2012)), the expression of genes encoding subunits of the immunoproteasome was also examined. Intriguingly, and in contrast to genes encoding MHC Class I subunits, low-level Hsp90i robustly induced the immunoproteasome genes Psme1, Psme2, Psmb8, Psmb9, and Psmb10 (FIG. 5A). Immunoproteasome beta subunits are homologous to constitutive proteasome subunits, but when incorporated into 20S core particles, the resultant immunoproteasomes generate distinct proteolytic products that exhibit high affinity for MHC Class I (Huber, E M. et al., Cell 148, 727-738, (2012).). Immunoblot analysis of heat-shock response marker (Hsp70), immunoproteasome subunit (Psmb8), constitutive proteasome subunit (Psmb5), total proteasome (20S), and loading control (Tubb) in MC38 cells treated with HSP90i for 24 or 72 hours at 0, 15, 30, 60, 120, and 250 nM concentration was carried out. Hsp90i increased the protein levels of Psmb8, the principal catalytic subunit of immunoproteasomes, and slightly decreased Psmb5 levels at 72 hours (FIG. 5B). Importantly, this effect was not observed at 24 hours post initiation of HSP90i, even at high concentrations, suggesting that induction of Psmb8 is not through the classical heat-shock response. The importance of the immunoproteasome in increasing MHC Class I expression in response to HSP90i is further supported by the observation that a Psmb8-specific inhibitor (ONX-0914) (Muchamuel, T. et al., Nat Med 15, 781-787, (2009)) abrogated the increase in MHC Class I surface expression caused by low level HSP90i treatment (FIG. 5C). Immunoblot analysis of lysate from MC38 cells treated with the 0, 15, 30, and 60 nM of HSP990 concurrent with DMSO or ONX-0914 was carried out. HSP90i induces Psmb8 in the presence of ONX-0914 demonstrating that the mechanism by which ONX-0914 blocks HSP90i-mediated MHC Class I induction is not through blocking induction of Psmb8.

MHC Class I peptide profiling has emerged as a powerful tool to study the antigen repertoire of tumor cells (Yadav, M. et al. Nature 515, 572-576 (2014)). To ascertain whether Hsp90i altered the antigenic profile of MC38 cells, MHC Class I immunoprecipitation was performed followed by peptide mass spectrometry of isolated antigens (represented schematically in FIG. 5D) (Khodadoust, M. S. et al., Nature 543, 723-727 (2017)). The peptides identified in all samples were of the expected size (8-11aa) and displayed amino acid preferences at known anchor residues for both MHC alleles (FIG. 5E, FIG. 5F, and FIG. 5G). 557 unique peptides from vehicle-treated MC38 cells were observed, and remarkably 1005 unique peptides were identified from Hsp90i-treated cells with 425 peptides identified in both groups (FIG. 5H, FIG. 5I, and, FIG. 5J). After sampling 5 technical replicates, identification of new peptides approached saturation indicating sufficient sampling of the cellular material (10 mg total protein) (FIG. 5K). In addition to an increased number of unique peptides observed with Hsp90i exposure, peptides common to both vehicle and Hsp90i-treated cells exhibited greater quantitative abundance in Hsp90i-treated samples (FIG. 5L). These findings provide direct experimental evidence that treatment with low-level Hsp90i diversifies and amplifies the antigen repertoire of tumor cells.

### Example 5: Anti-Tumor Effect of Low Dose Administration of HSP90i is Dependent on MHC Class I Expression

### Methods

B2m Knockout

For CRISPR/Cas9-mediated disruption of B2m, pSpCas9-P2A-GFP plasmid (Addgene #48138) was modified with a control sgRNA sequence (GTATTACTGATATTGGTGGG) or an sgRNA targeting B2m (TTGAATTTGAGGGGTTTCTG). Log phase MC38 cells were transfected with Ctrl or B2m plasmids using Lipofectamine 2000 (Invitrogen) according to manufacturer's instructions. 24 hours later, single GFP positive cells were sorted into 96-well plates to isolate individual MC38 clones with the desired modification. After 2 weeks, isolated clones were tested by western blot to confirm loss of B2m expression. Ctrl and B2m clones were then passaged through syngeneic mice (C57/B16) to increase the uniformity of tumor establishment and progression in subsequent experiments.

### Results

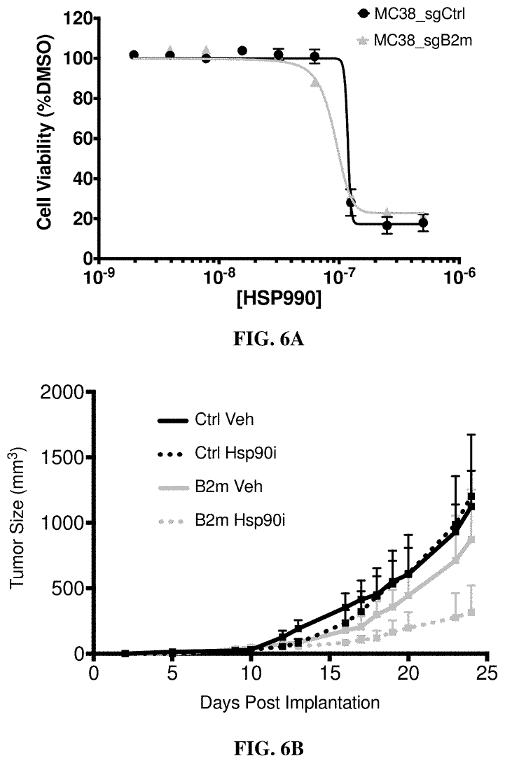

Collectively, the observations led to a model in which subtoxic HSP90 inhibition elicits an anti-tumor immune response by enhancing antigen presentation. Enhancement is mediated through increased expression of immunoproteasome components, presumably generating additional peptides that stabilize surface expression of MHC Class I. If correct, this model predicts that deletion of MHC Class I from the surface of MC38 cells should ablate the antitumor activity of low level HSP90 inhibition in vivo. To test the model, CRISPR/Cas9 was utilized to knockout the beta-2 microglobulin gene (B2m), causing destabilization of the MHC Class I heterocomplex and preventing its trafficking to the cell surface (Gettinger, S. et al., Cancer Discov 7, 1420-1435 (2017)). MC38 cells transfected with a control guide RNA (Ctrl) exhibited robust MHC Class I surface staining, whereas cells transfected with a guide targeting B2m (B2m KO) displayed no detectable MHC Class I on the cell surface (FIG. 6A and FIG. 6B). The Ctrl and B2m MC38 cells were equally sensitive to HSP90i indicating that inhibition of tumor progression in mice is not mediated by cell-intrinsic mechanisms. The Ctrl and B2m MC38 cells showed no difference in sensitivity to HSP90i in culture (FIG. 6C) and formed tumors, which grew with similar kinetics to that of the parental cells (FIG. 6D solid lines). Likewise, low-dose Hsp90i treatment reduced the growth of Ctrl tumors to a similar extent as parental MC38 cells (FIG. 3C). Strikingly, however, MC38 cells lacking MHC Class I (B2m KO) exhibited nearly identical growth kinetics when treated with HSP90i or vehicle control (FIG. 6C). These results indicate that continuous low dose administration of HSP90i elicits an anti-tumor effect that is dependent on MHC Class I expression on the surface of tumor cells.

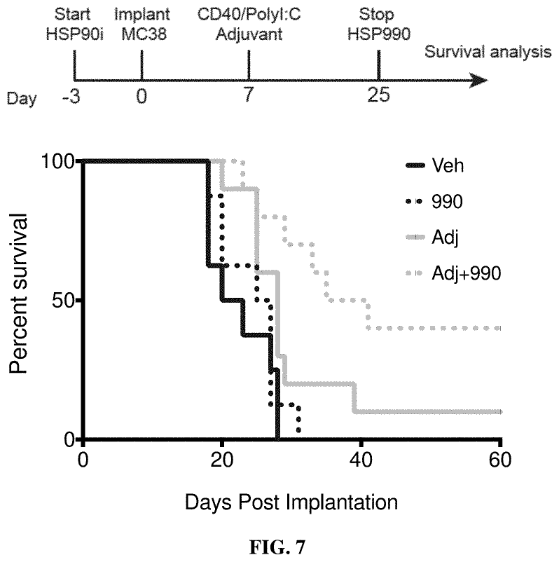

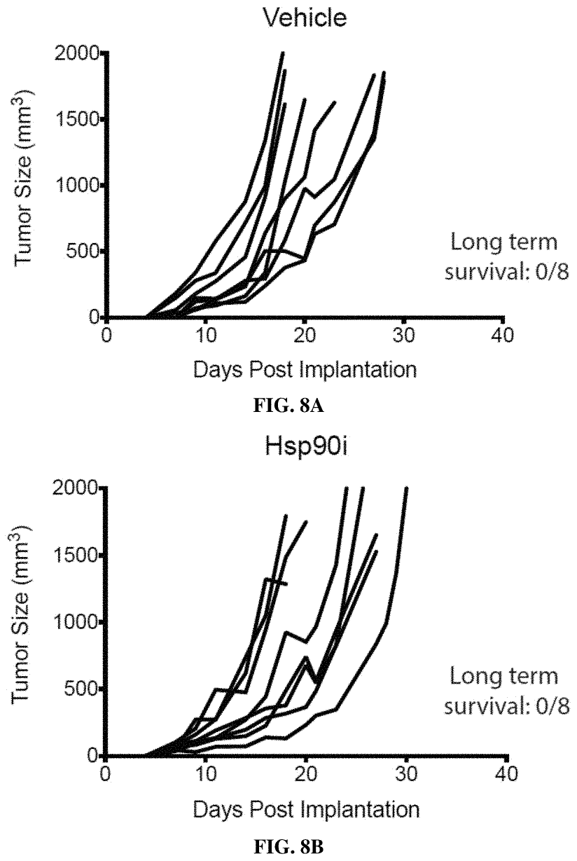

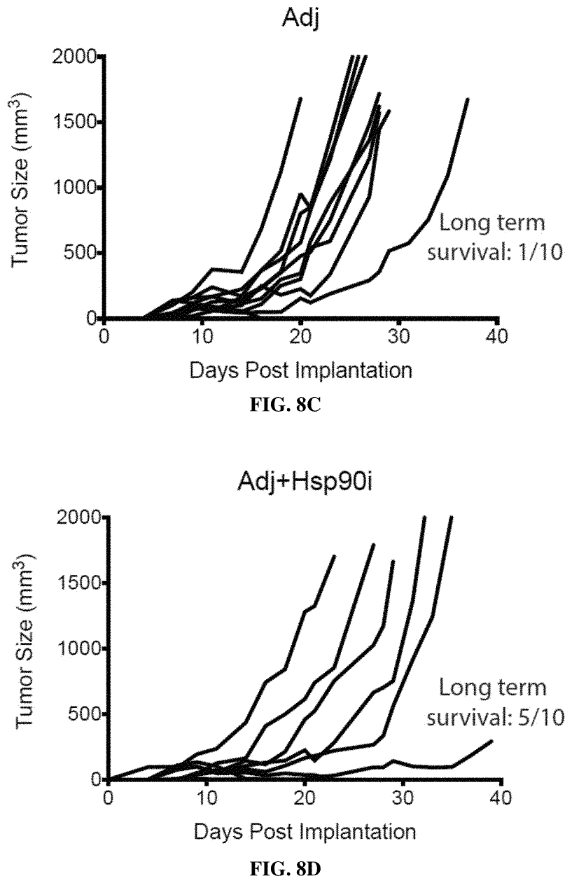

Whether non-specific stimulants of the immune system could enhance the effects of HSP90i treatment to increase antitumor activity was assessed. Treatment of mice carrying MC38 tumors with a combination of continuous low dose HSP90i and a single dose of conventional adjuvant cocktail (anti-CD40 and polyI:C) (Yadav, M. et al., Nature 515, 572-576 (2014); Llopiz, D. et al., Cancer Immunol Immunother 57, 19-29, (2008)) resulted in remarkably increased long-term survival (4 of 10 mice after 2 months) compared with either agent alone (FIG. 7, FIG. 8A, FIG. 8B, FIG. 8C, and FIG. 8D).

### Example 6: HSP90i Effects on MC38 Cells are Mechanistically Distinct from IFN-γ Effects

### Methods

Cell Culture MC38 cells were kindly provided by A. Sharpe from Dana Farber Cancer Institute. Cells were maintained in DMEM supplemented with 10% FBS and penicillin/streptomycin. Cells were confirmed negative for mycoplasma contamination by PCR-based assay. MC38 cells were seeded in 12 well plates at a density of 125,000 cells per well and allowed to attach for 24 hours, and then treated with the indicated concentrations of HSP90i (NVP-HSP990) or IFN-γ. For Ifngra knockout, control or sgRNA targeting Ifngra (TATGTGGAGCATAACCGGAG) were cloned into pLenti-CRISPRv2 (Addgene #52561). Lentivirus was produced with HEK293T cells and MC38 cells were transduced with standard protocols. Transduced cells were selected with 2 μg/mL puromycin for 72 hours. Selected cells were then treated with DMSO, 60 nM Hsp90i, 10 ng/mL Ifng, or the combination and analyzed by flow cytometry.

Flow Cytometry

For analysis of MHC Class I surface expression, cells were seeded into 12 well plates (125,000/well). 24-hours later, cells were treated with the indicated concentrations of HSP90i (NVP-HSP990) or DMSO vehicle. 72-hours after treatment, cells were dissociated with Accumax (Innovative Cell Technologies) and each treatment condition was distributed into 2 wells of a 96 well plate. Cells were spun at 500×g for 3 min, washed 1× in ice cold PBS supplemented with 2 mM EDTA and 0.5% FBS, and incubated with APC labeled anti-H2-Kb antibody (Clone AF6-88.5, Biolegend), PE-Cy7 labeled anti-PD-L1 antibody (Clone 10F.9G2), BV421 labeled Ifngr antibody (GR20), at a 1:400 dilution for 45 min on ice. After incubation with antibody, cells were washed 1× in ice cold PBS+10% FBS prior to analysis on a LSR-2 flow cytometer. Cytometric data were plotted and analyzed with FloJo.

Immunoblotting

Immunoblotting was performed as described in Example 4. Briefly, protein extraction was performed by washing cells with PBS and then isolating whole cell lysates in lysis buffer (50 mM Tris pH 7.5, 150 mM NaCl, 1 mM EDTA, 1% Triton-X100, 0.1% SDS, 1× Halt Protease/Phosphatase inhibitor) Lysates were then cleared by centrifugation and quantified using BCA Assay. Lysates were separated by SDS-PAGE and transferred to 0.2 μm nitrocellulose membranes prior to washing with PBST, blocking with 5% milk in PBST, and incubation with primary antibody overnight at 4° C. All primary antibodies were diluted 1:1000 in 2.5% milk in PBST. Membranes were then washed and incubated with HRP conjugated secondary antibody. Detection of secondary antibody was performed with SuperSignal West Pico or Femto ECL reagents (Pierce) and digitally imaged on a ChemiDoc (BioRad).

qRT-PCR

qRT-PCR was performed as described in Example 3. Briefly, total RNA isolation from cultured cells was performed using RNeasy kits (Qiagen) according to the manufacturer's instructions. Eluted RNA was treated with TURBO DNase (Ambion) and quantified by Nanodrop. 1.5 μg total RNA was then used for reverse transcription using the High Capacity cDNA Reverse Transcription Kit (Applied Biosystems) with random hexamers as primers. cDNA was diluted prior to PCR amplification with PowerUp SYBR Green Master Mix (Applied BioSystems) in a QuantStudio6 Real Time PCR System (Applied Biosystems) according to manufacturer's instructions.

### Results

To better understand the mechanism(s) by which HSP90 inhibition elicits an anti-tumor immune response to MC38 cells, further cell culture experiments were performed.

First, to confirm the results observed with NVP-HSP990, the effects of another HSP90i (SNX-2112) was evaluated in the MC38 cell culture model. Flow cytometry analysis of MHC-I surface expression demonstrated that both NVP-HSP990 and SNX-2112 induced MHC-I at the dosages tested (60 nM for each drug; data not shown). These results show that multiple, chemically distinct, orally bioavailable HSP90i(s) induce MHC-I at subtoxic doses.

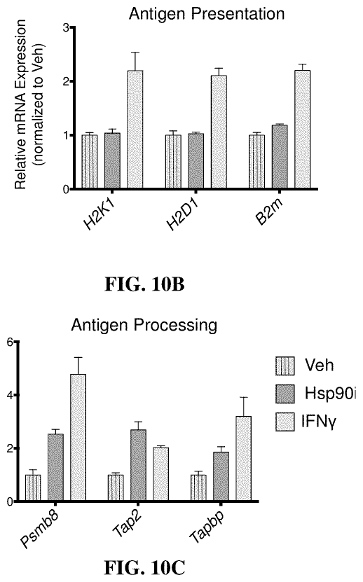

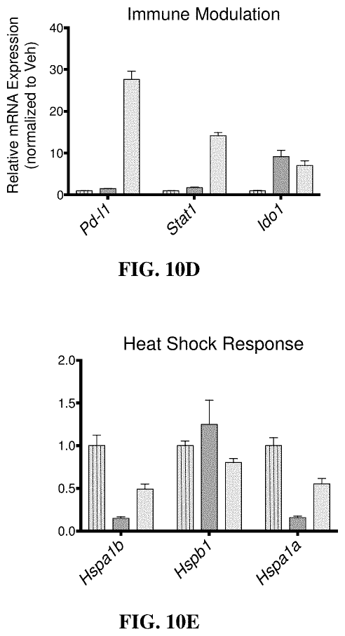

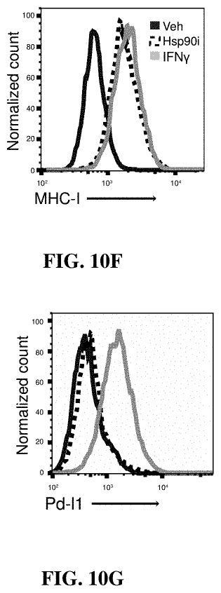

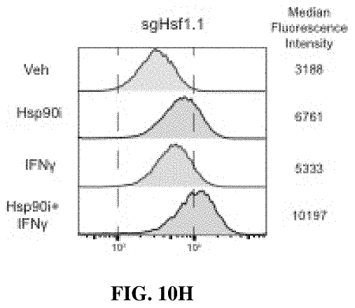

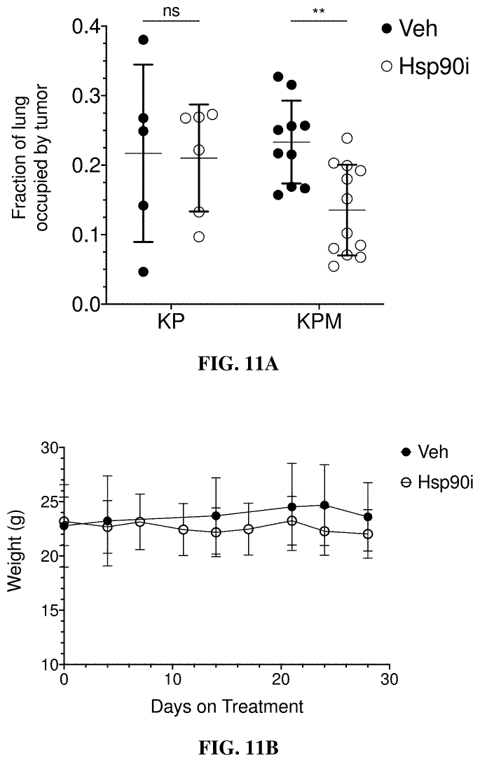

Given that IFN-γ is known to stimulate antigen presentation, the effects of HSP90 inhibition were compared to IFN-γ treatment. Exposure to IFN-γ is the canonical method by which non-immune cells induce antigen presentation, and alterations in IFN-γ signaling are known to influence immune escape in cancers. MHC-1 peptide profiling after Hsp90i (NVP-HSP990) or IFN-γ treatment showed that HSP90i induced a distinct repertoire of MHC-I peptides compared to IFNγ (FIG. 10A). This provides further support that Hsp90i diversifies the antigen repertoire of tumor cells. In addition to the peptides that are unique to Hsp90i treated cells, peptides that are enriched on surface MHC-I molecules in IFNγ treated cells were also found to be enriched in Hsp90i treated cells (not shown).

QRT-PCR analysis of antigen processing and presentation genes, immune checkpoint genes, and heat shock inducible genes in MC38 cells following treatment with Hsp90i (60 nM) or IFN-γ (10 ng/mL) showed that the effect of Hsp90i was largely distinct from IFN-γ (FIG. 10B-E). Immunoblot analysis showed that IFN-γ induced expression of downstream markers of IFN-γ signaling such as Stat1, Stat3, pStat3, and PD-11, while HSP90i exhibited a minimal to undetectable effect (not shown). These results indicate that Hsp90i engages a transcriptional program that is mechanistically distinct from IFN-γ, which avoids activation of the immunosuppressive ligand PD-L1 and the cytoprotective heat shock response.

Flow cytometry analysis of treated cells showed that Hsp90i stimulated MHC-I presentation in a distinct manner compared to IFN-γ (FIG. 10F, 10G). Flow cytometry analysis further illustrated that upon Cas9/sgRNA mediated disruption of the alpha chain of the IFN-γ receptor, Hsp90i induced MHC-I in IFN-γ receptor KO cells and rescued the lack of antigen presentation following IFN-γ treatment (FIG. 10H). As disruptions and/or mutations in IFN-γ signaling are known to confer resistance to immunotherapy, Hsp90i may represent a much needed way to boost immune responses in these patient populations.

Finally, to evaluate the role of heat shock response on the induction of MHC-I post HSP90i treatment, HSF1 knockout MC38 cells were generated and evaluated. As shown in FIG. 10H, the heat shock responsive transcription factor HSF1 was not required for the induction of MHC-I following Hsp90i. Results were consistent across two different Cas9/sgRNA generated knockout cell lines. This data demonstrates that the mechanism of Hsp90i stimulating immune responses is not through a classical heat shock response.

### Example 7: HSP90i Reduces Tumor Burden In Vivo in Tumors with High Mutational Burden

### Methods

Mice

A genetically engineered mouse model of non-small cell lung cancer (NSCLC) was used. Mice harboring a Cre-recombinase inducible oncogenic Kras, and a floxed p53 allele (KP) develop tumors following intratracheal administration of Cre recombinase. In addition, a hypermutant mouse model of NSCLC (KPM) was used in which mice have a floxed allele of the MutS homolog 2 (Msh2) gene, a critical component of DNA mismatch repair, in addition to the genetic modifications of the KP mouse model. These models enable the interrogation of Hsp90i strategies in a physiologically relevant NSCLC model with and without hypermutation.

Treatment

KP and KPM mice were infected intratracheally with Adenovirus expressing Cre recombinase from an SPC promoter at a titer of 2×10⁸ plaque forming units per mouse to initiate tumor formation. 8 weeks following tumor initiation, the cohort was randomized and vehicle (control) or Hsp90i (NVP-HSP990) was administered in the drinking water at a dose of 0.5 mg/kg/day for 4 weeks prior to analysis at 12 weeks post tumor induction.

Tumor Burden Analysis

Tumor burden was analyzed histologically on tumor sections stained with hemotoxylin and eosin (H&E) using standard protocols at the Koch Institute Histology Facility. Tumor burden was calculated by dividing the total area of tumor tissue over the total area of normal lung tissue present in each sample.

### Results

To extend the above results to an in vivo setting, the effects of HSP90 inhibition on tumor burden was evaluated in two mouse models of non-small cell lung cancer. These mouse models enable the interrogation of HSP90i strategies in a physiologically relevant NSCLC model with and without hypermutation.

After four weeks of treatment with HSP90 inhibition, no significant difference was observed in the lung tumor burden of vehicle treated and HSP90i treated KP mice (FIG. 11A). However, in KPM mice, HSP90i significantly reduced the lung tumor burden compared to control (FIG. 11A). Notably, four weeks of low dose HSP90i treatment showed minimal effects on mouse mass or body condition indicating that this strategy exhibited very little, if any, systemic toxicities (FIG. 11B).

These results show that HSP90i can stimulate an anti-tumor immune response when a sufficient or high amount of neoantigens are present. Notably, the majority of NSCLC patients present with a high degree of mutational burden indicating that this strategy could be broadly useful in lung cancer.

### Summary

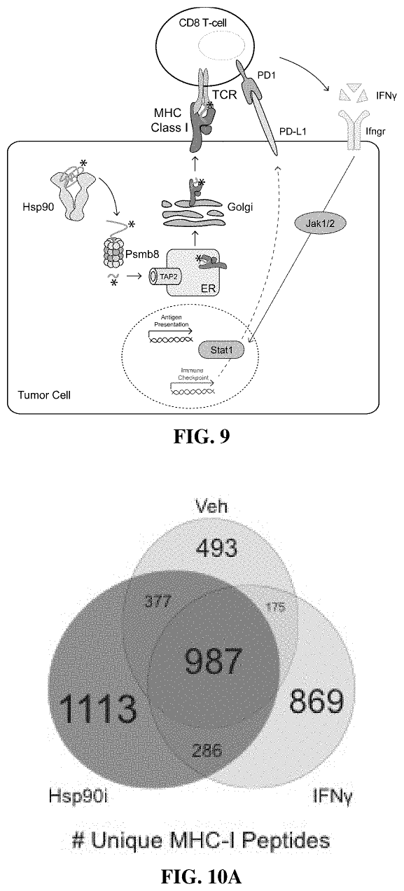

Based on the experiments and data described in the above Examples, the following model is proposed for HSP90i-enhanced antigen presentation and anti-tumor immunity (FIG. 9). Limiting the buffering capacity of HSP90 drives the degradation of mutant, neoantigen-containing proteins (asterisk in FIG. 9) via the immunoproteasome. The resulting peptides are subsequently loaded onto MHC-I for presentation in a manner that is distinct from classical IFN-γ signaling through Jak/Stat pathways. Increased substrate load clearly contributes to additional peptides entering the antigen presentation pathway, thereby diversifying the peptide repertoire presented by tumor cells. By avoiding canonical IFN-γ signaling, HSP90i can stimulate MHC-I presentation while avoiding the immunosuppression associated with PD-L1 expression.

HSP90 acts as a protein-folding buffer that shapes the manifestations of genetic variation in model organisms (Queitsch, C. et al., Nature 417, 618-624 (2002); Rutherford, S L et al., Nature 396, 336-342 (1998).) and in man (Karras, G. I. et al., Cell 168, 856-866 (2017)). It has been shown that targeting this ancient role of HSP90 could provide a unique way to expose the “otherness” of genetically unstable, highly malignant cancers by revealing their aberrant proteome to the immune system in the context of MHC Class I (Ott, P. A. et al., Nature 547, 217-221 (2017)). Limiting the buffering capacity of HSP90 induces the degradation of mutant proteins containing neoantigens via the immunoproteasome and subsequent loading onto MHC Class I for presentation to T-cells (FIG. 9). Thus, anti-tumor activity can be achieved with low-dose HSP90 inhibition via a cell non-autonomous, immune-mediated mechanism at concentrations that are 20-fold lower than the peak plasma concentration of the Phase-2 recommended dose of HSP9908.

Beyond the new biological insights provided, these results support the clinical re-evaluation of orally bioavailable HSP90i(s), such as NVP237 HSP990, given on a sustained low-dose schedule of administration with the intent to enhance tumor immunogenicity, with or without a concurrent adjuvant. As orally bio-available HSP90i(s) such as NVP-HSP990 and SNX-5422 have already been studied in Phase I and Phase II trials, their pharmacokinetic and safety profiles in humans are well established. Re-purposing the extensive development work that has already gone into such compounds could allow for the very rapid clinical testing of HSP90 inhibition as a new, mechanistically distinct immunotherapeutic strategy for treating cancers.

Unless defined otherwise, all technical and scientific terms used have the same meanings as commonly understood by one of skill in the art to which the disclosed invention belongs. Publications cited and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A kit or pharmaceutical composition comprising an effective amount of the combination of one or more HSP90 inhibitor(s) and one or more immunostimulatory agent(s),
wherein the amount of the HSP90 inhibitor(s) does not induce systemic toxicity, and
wherein administration of the pharmaceutical composition reduces cancer cell proliferation or reduces cancer cell viability, or reduces both cancer cell viability and proliferation in a subject with cancer to a greater degree than administering to the subject the same amount of HSP90 inhibitor(s) alone or the same amount of immunostimulatory agent(s) alone.

2. The kit or pharmaceutical composition of claim 1, wherein the reduction in cancer cell proliferation or viability in the subject with cancer is more than the additive reduction achieved by administering the HSP90 inhibitor(s) alone or the immunostimulatory agent(s) alone.

3. The kit or pharmaceutical composition of claim 1, wherein the one or more HSP90 inhibitors is of a class selected from the group consisting of Benzoquinone Ansamycin Antibiotics, resorcinol derivatives, purine scaffold HSP90 inhibitors, functional nucleic acid inhibitors, and inhibitor of the expression or function of one or more co-chaperones.

4. The kit or pharmaceutical composition of claim 3, wherein the one or more HSP90 inhibitors is selected from the group consisting of geldanamycin (GA), 17-Allylamino-17-demethoxy-geldanamycin (17-AAG), 17-dimethylaminoethylamino-17-demethoxy-geldanamycin (17-DMAG), and IPI-504 (Retaspimycin).

5. The kit or pharmaceutical composition of claim 3, wherein the one or more HSP90 inhibitors is selected from the group consisting of AUY922, AT13387 (Onalespib), KW-2478, and STA-9090 (ganetespib).

6. The kit or pharmaceutical composition of claim 3, wherein the one or more HSP90 inhibitors is selected from the group consisting of Debi0932, PUH71, CNF-2024/BIIB021, MPC-3100 and BIIB021.

7. The kit or pharmaceutical composition of claim 1, wherein the one or more HSP90 inhibitors is selected from the group consisting of TAS-116, Radicicol, Radanamycin, DS-2248, XL-888, NMS-E973, NVP-HSP990, Rifabutin, and SNX-5422.

8. The kit or pharmaceutical composition of claim 3, wherein one or more inhibitors of the expression or function of one or more co-chaperones is selected from the group consisting of Hsp70, Hsp27, HSF-1, Hop and p23.

9. The kit or pharmaceutical composition of claim 1, wherein the one or more immunostimulatory agents is of a class selected from the group consisting of pro-inflammatory molecules, adjuvants, tumor antigens, antagonists of immuno-suppressors, modulators or regulatory T cells (T-regs) and co-stimulatory antibodies.

10. The kit or pharmaceutical composition of claim 9, wherein the one or more immunostimulatory agents is an antagonist of an immuno-suppressor molecule selected from the group consisting of CTLA-4, PD-1, PD-L1, PD-L2, IL-10, and TGF-β.

11. The kit or pharmaceutical composition of claim 10, wherein the one or more immunostimulatory agents is a monoclonal antibody.

12. The kit or pharmaceutical composition of claim 11, wherein the dosage of the monoclonal antibody is between 1 mg/kg and 15 mg/kg body weight of the recipient.

13. The kit or pharmaceutical composition of claim 12, wherein the monoclonal antibody is an anti-PD-L1 antibody.

14. The kit or pharmaceutical composition of claim 1, wherein the dosage of the HSP90 inhibitor is between 1% and 50% of the amount that is the maximum tolerated dose (MTD) in humans.

15. The kit or pharmaceutical composition of claim 1, wherein the dosage of the HSP90 inhibitor is 5% of the amount that is the maximum tolerated dose (MTD) in humans.

16. The kit or pharmaceutical composition of claim 1 further comprising an additional active agent.

17. A method of treating one or more symptoms of a cancer in a human patient comprising administering to the patient an effective amount of one or more HSP90 inhibitor(s) in combination with an effective amount of one or more immunostimulatory agent(s) as defined by claim 1,
wherein administration of the combination of one or more HSP90 inhibitor(s) and one or more immunostimulatory agent(s) reduces cancer cell proliferation or viability in the patient to a greater degree than administering to the patient the same amount of HSP90 inhibitor(s) or the same amount of immunostimulatory agent(s) alone.

18. The method of claim 17, wherein the reduction in cancer cell proliferation or viability in the subject with cancer is more than the additive reduction achieved by administering the HSP90 inhibitor(s) and or the same amount of immunostimulatory agent(s) alone.

19. The method of claim 17, wherein the amount of HSP90 inhibitor(s) does not affect the cancer cells when the HSP90 inhibitor(s) is administered without co-administration of the immunostimulatory agent(s).

20. The method of claim 17, wherein the one or more immunostimulatory agent(s) is administered to the human prior to administration of one or more HSP90 inhibitor(s) to the patient.

21. The method of claim 17, wherein the one or more HSP90 inhibitor(s) is administered to the human prior to administration of one or more immunostimulatory agents to the subject.

22. The method of claim 17, further comprising administering to the human one or more additional active agents.

23. The method of claim 22, wherein the one or more additional active agents is an anti-cancer agent.

24. The method of claim 17 further comprising administering to the human surgery or radiation therapy.

25. The method of claim 17, wherein the cancer is selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, skin cancer, liver cancer, melanoma, squamous cell carcinomas of the mouth, throat, larynx, and lung, colon cancer, cervical cancer, cervical carcinoma, epithelial cancer, renal cancer, genitourinary cancer, pulmonary cancer, esophageal carcinoma, head and neck carcinoma, large bowel cancer, hematopoietic cancers; testicular cancer; colon and rectal cancers, and pancreatic cancer.

26. The method of claim 17, wherein the HSP90 inhibitor is administered daily.

27. The method of claim 17, wherein the HSP90 inhibitor is NVP-HSP990, administered with daily oral dosing of 2.5 mg, to achieve a steady state drug concentration of 20-40 nM in plasma.

28. The method of claim 17, wherein the cancer has a high mutational burden.
